SEQUENCE LISTING (6 sequences)

SEQ ID NO: 1 - DNA, 44 bp, artificial
  Primer CD80F
ttcaagcttaccatgggccacacacggaggcagggaacatcacc

SEQ ID NO: 2 - DNA, 29 bp, artificial
  Primer hB7-1R
taatctagatgcggacactgttatacagg

SEQ ID NO: 3 - protein, 9 aa, Homo sapiens
GLCTLVAML

SEQ ID NO: 4 - protein, 10 aa, Homo sapiens
ELAGIGILTV

SEQ ID NO: 5 - DNA, 41 bp, Artificial
  Primer CD86F
taagctagcaccatggatccccagtgcactatgggactgag

SEQ ID NO: 6 - DNA, 38 bp, Artificial
  Primer CD86R
taactcgagttaaaaacatgtatcacttttgtcgcatg

(19) United States
(12) Patent Application Publication
(10) Pub. No.: US 2005/0048646 A1
(43) Pub. Date: Mar. 3, 2005

(54) Method for inducing cytotoxic T lymphocyte

(75) Inventors: Mie Nieda, Setagaya-ku (JP); Katsuo Noguchi, Setagaya-ku (JP); Toshimasa Tadaki, Setagaya-ku (JP); Shigemi Sasawatari, Setagaya-ku (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: MEDINET Co., Ltd., Yokohama-shi (JP)

(21) Appl. No.: 10/901,067

(22) Filed: Jul. 29, 2004

(30) Foreign Application Priority Data
Aug. 25, 2003 (JP) 2003-341822
Jan. 28, 2004 (JP) 2004-020436

(51) Int. Cl. A61K 38/08; C12N 5/08

(52) U.S. Cl. 435/372; 514/16

(57) ABSTRACT

A method for inducing an antigen-specific cytotoxic T lymphocyte (CTL) which recognizes determinants of disease-related antigens, such as from cancer or tumor cells, viral infections, intracellular bacterial infections, or parasitic infections. Lymphocytes are contacted or co-cultured with a cell line which expresses at least one major histocompatibility (MHC) class I molecules and a co-stimulatory molecule, after or while bringing the cell line into contact with a disease antigenic peptide. Alternatively, the lymphocytes may be contacted with a cell line expressing at least one MHC class I molecules and an accessory molecule and presenting an antigen. The CTLs induced by these methods may be used to treat cancer or infectious diseases. Methods for testing the proliferation potential of a lymphocyte sample using cell lines expressing MHC class I molecules and accessory molecules are also disclosed.

### BACKGROUND OF THE INVENTION

1. Field of the Invention

The present invention relates to a method for inducing CTLs (cytotoxic T lymphocytes) by co-culturing of lymphocytes with a cell line transformed with and expressed co-stimulatory molecules, such as CD80 (B7.1) or CD86 (B7.2). Further, the present invention relates to CTLs induced by the above-mentioned method and uses thereof such as treatment or prevention for a disease, methods of preventing or treating diseases such as infectious diseases and cancers, and a method for testing the ability of lymphocytes from a subject to determine their responsiveness to the transformed cell line.

2. Description of Related Art

Cytotoxic T cells generally recognize antigens directly, but also recognize processed antigens that are presented in the context of an MHC class I heterodimer responsiveness a processed antigenic peptide, generally about 8-12 amino acid residues in length, binds. A cytotoxic lymphocyte will recognize the processed antigenic peptide when bound and presented in the context of the MHC class I molecules. Other accessory or co-stimulatory molecules can augment or facilitate the recognition of the processed peptide by cytotoxic T cells and subsequent activation and proliferation of the cytotoxic T cells.

T cell mediated immunotherapy has been attempted for a variety of different diseases including viral diseases, diseases caused by intracellular bacteria, fungal or parasitic diseases, and cancer.

Cancer is a disease caused by various factors, and therapeutic methods thereof that have been employed so far include abscission, radiation and administration of an anti-tumor agent. Besides these conventional methods, various studies recently have been carried out for resolving the mechanism of cancer crises at levels of genes and proteins by using a genetic engineering technique such as gene diagnosis and therapy so as to diagnose and treat cancers more precisely. Among these techniques, there is a cell-mediated immunotherapy.

In the cell-mediated immunotherapy, in order to eradicate lesions diagnosed as cancer by immunocompetent cells (lymphocytes, dendritic cells and the like), the functions of immunocompetent cell artificially are strengthened considerably, thus treating cancer. More specifically, immunocompetent cells of a patient are harvested from peripheral blood (PB) or stem cells and then activated and proliferated. Thereafter, these immunocompetent cells are injected into the patient, thereby increasing the immune response of the patient so as to regress and eradicate cancer or arrest and inhibit the proliferation of cancer. For infectious diseases, it also is possible to treat infected cells in a similar manner.

The cell-mediated immunotherapy includes a CD3-LAK (CD3 lymphokine-activated killer) therapy, a CTL (cytotoxic lymphocytes) therapy, a TIL (tumor-infiltrating lymphocytes) therapy, a DC (dendritic cell) therapy and the like.

By using an anti-CD3 antibody and an IL-2 to activate lymphocytes, the CD3-LAK therapy provides a nonspecific activity which is a cytotoxic activity to cells infected with virus or the like or cells mutated into cancer (in the following, referred to as mutated cells) in general.

The CTL, TIL and DC therapies selectively induce lymphocytes, in particular CTLs having a specific cytotoxic activity against cancer cells/cancers or infected cells in the patients with cancer or infectious disease. Since these CTLs act specifically on mutated cells of a patient, they are considered to be particularly effective among the cell-mediated immunotherapies.

In the CTL therapy, APCs (antigen-presenting cells) or mutated cells as stimulator cells and lymphocytes as responder cells that are harvested from a patient are co-cultured ex vivo, thus inducing CTLs. In the TIL therapy, lymphocytes in the vicinity of cancer tissue are harvested, and CTLs contained therein are activated and induced.

Although CTLs induced by the above-described methods are effective among the cell-mediated immunotherapies, attempts have been made to further enhance effectiveness by CTL induction using DCs as APCs. The DCs, which can express or present antigens derived from lung cancer, hepatoma, myeloma, lymphoma, pancreatic cancer or the like or specific antigenic peptides derived from these cancers on the surface, are co-cultured with lymphocytes, thereby inducing CTLs functioning specifically to a certain tumor associated antigen (see JP 2003-242 A, for example).

In JP 2003-242 A, in order to induce the specific CTLs, major histocompatibility antigen (HLA-A24) restricted prostate cancer antigenic peptides are brought into contact with antigen-presenting cells such as DCs, followed by co-culturing with lymphocytes, thereby inducing prostate cancer-specific CTLs. However, prior art methods for inducing CTLs have the following problems.

In many CTL inductions ex vivo for CTL therapies, it is necessary to use cancer cells that are harvested from patients when the cancer cells are used as APCs. In addition in general the cancer cells don't work effectively as APCs because of loss of co-stimulatory molecules and accessory molecules. In the TIL (tumor-infiltrating lymphocytes) therapy, it is not always that cancer cells are available. For example, it can be harvested only on limited occasions, such as during an operation. DCs are professional APCs which can efficiently process and present tumor-associated antigens derived from cancers. Therefore, to use DCs is the most efficient tool for the induction of CTL ex vivo. However, the preparation of DCs ex vivo needs very complicated process steps by using variety of cytokines which are very expensive and can be completed by scientists with high quality skill in addition of time consuming. Moreover, it is very hard to reproducibly prepare DCs which present antigens in a stable or consistent manner in particular from patients since a large number of PB cells or stem cells are required to prepare enough DCs.

To attempt to solve these problems, methods have also been developed which involve expression of high levels of CD80 on cancer cells. CD80 (B7.1) is a co-stimulatory molecule present on mature dendritic cells and is involved in activating CTLs. The B7 ligands CD80 (B7.1) and CD86 (B7.2) on APCs can deliver either co-stimulatory or inhibitory signals to the T cell when interacting with their respective ligands CD28 and CD 152 (CTLA-4) on the T cell surface. Cancer cells expressing the high levels of CD80 and lymphocytes can be co-cultured, thereby inducing CTLs (for example, see Grant-in-Aid from the Ministry of Science, Education and Culture of Japan, Report, 1995-1996; Grant number 07457150). Furthermore, a technology has been developed in which B7 molecules are expressed at high levels on tumor cell lines, thereby inducing CTLs or NK (natural killer) cells (see JP 01-029191 A1, for example). However, these methods require producing specific CD80-expressing tumor or cancer cell lines each time the methods are performed, and thus do not provide standard, reliable cell lines for the expression of tumor or cancer related antigens or antigenic peptides. Also, in JP 01-029191 A1, although B7 molecules are expressed at a high level on the cell lines, whether the actual molecules are CD80 (B7.1) or CD86 (B7.2) is unknown because the derivation of B7 genes is not clear. Furthermore, since the cell lines in these methods either lack MHC class I antigen or have lowered expression of MHC class I molecules, antigen-specific CTLs restricted in MHC class I molecules cannot be induced effectively with such cell lines. Moreover, as these methods involve specific tumor cell lines, they do not address the problems involved in treatment of other types of cancer. For example, in order to induce CTLs specific for a particular cancer, it is necessary to produce a cell line derived from that particular cancer cell or a cell expressing that particular cancer antigen on its surface. In order to induce CTLs specific to each cancer, it is necessary to produce a cell line derived from that cancers or expressing that tumor/cancer-associated antigen on its surface and such cells do not generally induce CTLs efficiently.

### SUMMARY OF THE INVENTION

In view of the problems discussed above, one object of the present invention is to provide a method for inducing antigen-specific CTLs capable of addressing antigens associated with specific disease in a simpler and more efficient manner. Other general objects of the invention include providing CTLs which can be used to treat cancer and other diseases. Other objects of the invention are described below.

(1) A method for inducing a CTL, comprising contacting a cell line that expresses at least one major histocompatibility antigen (MHC) class I molecule and which has been transformed with at least one co-stimulatory molecule with an isolated or purified antigenic peptide and with a lymphocyte for a time and under conditions suitable for inducing a CTL specific for said antigenic peptide; (2) the method for inducing a CTL according to (1), wherein said contacting occurs in vitro; (3) the method for inducing a CTL according to (1), wherein said cell line is a human cell line, which expresses at least one MHC class I antigen heavy chain and β2-microglobulin, and at least one co-stimulatory or accessory molecules such as adhesion molecules; (4) the method for inducing a CTL according to (1), wherein said cell line is a stomach cancer-derived JR-st, a renal cancer-derived TUHR10TKB or a breast cancer-derived MDA-MB-231; (5) the method for inducing a CTL according to (1), wherein said cell line has been transformed with a nucleic acid expressing CD80 (B7.1); (6) the method for inducing a CTL according to (5), wherein said cell line expresses CD80, CD54 (ICAM-1) and CD40; (7) the method for inducing a CTL according to (1), wherein said cell line has been transformed with a nucleic acid encoding CD86 (B7.2); (8) the method for inducing a CTL according to (7), wherein said cell line expresses CD86 (B7.2), CD54 and CD40; (9) the method for inducing a CTL according to (1), wherein said antigenic peptide consists of a peptide having 8-11 amino acid residues; (10) the method for inducing a CTL according to (1), wherein said antigenic peptide corresponds to a portion of a cancer antigen; (11) the method for inducing a CTL according to (1), wherein said antigenic peptide corresponds to a portion of an antigen associated with an infectious disease; (12) the method for inducing a CTL according to (1), wherein said cell line expresses a class I the MHC which is HLA-A2 or HLA-A24, or both; (13) the method for inducing a CTL according to (1), wherein said cell line is homozygous for HLA-A2; (14) the method for inducing a CTL according to (1), wherein said cell line is homozygous for HLA-A*0201; (15) the method for inducing a CTL according to (1), wherein said cell line is homozygous for HLA-A24; (16) the method for inducing a CTL according to (1), wherein said cell line is homozygous for HLA-A*2401; (17) the method for inducing a CTL according to (1), wherein said cell line is homozygous for HLA-A*2402; (18) the method for inducing a CTL according to (1), wherein said lymphocyte is an autologous lymphocyte or an allogeneic lymphocyte that shares with at least one MHC class I molecule on said cell line;

(19) An antigen-specific CTL produced by the method for inducing a CTL according to (1); (20) an antigen-specific CTL produced by the method for inducing a CTL according to (4); (21) an antigen-specific CTL produced by the method for inducing a CTL according to (5); (22) an antigen-specific CTL produced by the method for inducing a CTL according to (7);

(23) A method for treating or preventing cancer or an infectious disease comprising administering to a subject in need thereof the CTL produced by the method for inducing a CTL according to (1); (24) a method for treating or preventing cancer or an infectious disease comprising administering to a subject in need thereof the CTL produced by the method for inducing a CTL according to (4); (25) the method for inducing a CTL according to (23), wherein said CTL is produced in vitro and is infused into said patient; (26) the method for inducing a CTL according to (23), wherein said disease is cancer and said CTL recognizes an antigenic determinant of an antigen associated with the cancer; (27) the method for inducing a CTL according to (23), wherein said disease is an infectious disease, and said CTL recognizes an antigenic determinant of an antigen associated with said infectious disease;

(28) A method for inducing a CTL, comprising contacting with a lymphocyte with a cell line that expresses at least one MHC class I molecule, an antigen and a co-stimulatory molecule for a time and under conditions suitable for induction of a CTL specific for said antigen, wherein said cell line has been transformed with at least one co-stimulatory molecule or exogenous antigen; (29) the method for inducing a CTL according to (28), wherein said contacting occurs in vitro; (30) the method for inducing a CTL according to (28), wherein said cell line is a human cell line, which expresses at least one MHC class I antigen heavy chain and β2-microglobulin, and at least one co-stimulatory or accessory molecules such as adhesion molecules; (31) the method for inducing a CTL according to (28), wherein said cell line is a stomach cancer-derived JR-st, a renal cancer-derived TUHR10TKB or a breast cancer-derived MDA-MB-231; (32) the method for inducing a CTL according to (28), wherein said cell line has been transformed with a nucleic acid expressing CD80 (B7.1); (33) the method for inducing a CTL according to (32), wherein said cell line expresses CD80, CD54 (ICAM-1) and CD40; (34) the method for inducing a CTL according to (28), wherein said cell line has been transformed with a nucleic acid encoding CD86 (B7.2); (35) the method for inducing a CTL according to (34), wherein said cell line expresses CD86 (B7.2), CD54 and CD40; (36) the method for inducing a CTL according to (28), wherein said antigen is a cancer antigen; (37) the method for inducing a CTL according to (28), wherein said antigen is an antigen associated with an infectious disease; (38) the method for inducing a CTL according to (28), wherein said cell line expresses a MHC class I which is HLA-A2 or HLA-A24, or both; (39) the method for inducing a CTL according to (28), wherein said cell line is homozygous for HLA-A2; (40) the method for inducing a CTL according to (28), wherein said cell line is homozygous for HLA-A*0201; (41) the method for inducing a CTL according to (28), wherein said cell line is homozygous for HLA-A24; (42) the method for inducing a CTL according to (28), wherein said cell line is homozygous for HLA-A*2401; (43) the method for inducing a CTL according to (28), wherein said cell line is homozygous for HLA-A*2402; (44) the method for inducing a CTL according to (28), wherein said lymphocyte is an autologous lymphocyte or an allogeneic lymphocyte that shares with at least one MHC class I molecule with said cell line;

(45) An antigen-specific CTL produced by the method for inducing a CTL according to (28); (46) an antigen-specific CTL produced by the method for inducing a CTL according to (31); (47) an antigen-specific CTL produced by the method for inducing a CTL according to (32); (48) an antigen-specific CTL produced by the method for inducing a CTL according to (34);

(49) A method for treating or preventing cancer or an infectious disease comprising administering to a subject in need thereof the CTL produced by the method for inducing a CTL according to (28); (50) a method for treating or preventing cancer or an infectious disease comprising administering to a subject in need thereof the CTL produced by the method for inducing a CTL according to (31); (51) the method for inducing a CTL according to (49), wherein said CTL is produced in vitro and is infused into said patient; (52) the method for inducing a CTL according to (49), wherein said disease is cancer and said CTL recognizes an antigenic determinant of an antigen associated with the cancer; (53) the method for inducing a CTL according to (49), wherein said disease is an infectious disease, and said CTL recognizes an antigenic determinant of an antigen associated with said infectious disease;

(54) A method for testing a proliferation potential of a CTL, comprising co-culturing a cell line that expresses at least one MHC class I molecule and a co-stimulatory molecule with a lymphocyte harvested from a subject, and simultaneously or subsequently bringing the cell line into contact with a disease antigenic peptide, thus determining the ability of said cell line and antigenic peptide to induce proliferation of a CTL; (55) the method for testing a proliferation potential of a CTL according to (54), wherein said cell line is a stomach cancer-derived JR-st, a renal cancer-derived TUHR10TKB or a breast cancer-derived MDA-MB-231; (56) the method for testing a proliferation potential of a CTL according to (54), wherein the co-stimulatory molecule on said cell line is at least one of CD80 and CD86; (57) the method for testing a proliferation potential of a CTL according to (54), wherein the type of the MHC class I of the cell line is HLA-A2 or HLA-A24, or both; (58) the method for testing a proliferation potential of a CTL according to (54), wherein the antigenic peptide is a cancer antigenic peptide or an infectious disease antigenic peptide.

(59) A method for testing a proliferation potential of a CTL, comprising co-culturing a cell line that expresses at least one MHC class I molecule with a lymphocyte harvested from a subject, a disease antigen and a co-stimulatory molecule; (60) the method for testing a proliferation potential of a CTL according to (59), wherein said cell line is a stomach cancer-derived JR-st, a renal cancer-derived TUHR10TKB or a breast cancer-derived MDA-MB-231; (61) the method for testing a proliferation potential of a CTL according to (59), wherein the co-stimulatory molecule on the cell line is at least one of CD80 and CD86; (62) the method for testing a proliferation potential of a CTL according to (59), wherein the type of the MHC class I of the cell line is HLA-A2 or HLA-A24, or both; and (63) the method for testing a proliferation potential of a CTL according to (59), wherein the antigen is a cancer antigen or an infectious disease antigen.

In conventional methods for inducing CTLs, attempts to induce CTLs by using DCs or cells expressing CD80 have been made. However, it has been necessary to induce DCs or prepare the cells expressing CD80 at every time, so that the processes thereof are complicated. Furthermore, DCs or other APCs cannot be obtained easily, making it difficult to induce CTLs in a stable manner.

With a view to inducing CTLs without such complicated conventional processes as described above, the inventors of the present invention searched for cells that can substitute for DCs in activating immuncometent cells such as lymphocytes. The inventors first established a cell line expressing co-stimulatory molecules and then confirmed that this cell line expressed various co-stimulatory molecules as well as accessory molecules such as adhesion molecules and MHC class I molecules on the cell surface. Using this cell line instead of DCs which is the most efficient APS for the induction of CTL, it was found that CTLs were induced constantly and stably, completing the present invention.

Moreover, at the time of co-culturing of the above-noted cell line with lymphocytes, by bringing a specific disease antigenic peptide, for example, a cancer antigenic peptide or an infectious disease antigenic peptide into contact therewith, it was found that CTLs specific for that disease antigen can be very efficiently induced.

The method for inducing a CTL according to the present invention makes it possible to induce CTLs specific for a certain disease antigen more promptly and simply than the conventional inducing method using APC including immature DCs and mature DCs. Also, the use of a cell line expressing co-stimulatory molecules constantly and stably improves reproducibility, allowing simpler and more precise induction of antigen-specific CTLs. Further, the inducing method of the present invention can induce CTLs more efficiently than the conventional method. Thus, in accordance with the present invention, utilizing the properties described above, it is possible to use efficiently induced CTLs for various purposes, for example, providing drugs or vaccines for cancers and viral infections and a therapeutically-effective cell-mediated immunotherapy. Accordingly, cancers, viral infections and the like can be treated more precisely than a conventional case.

### BRIEF DESCRIPTION OF THE DRAWINGS

FIG. 1A shows an example of the result of SDS-PAGE after immunoprecipitation, and FIG. 1B illustrates an example of the result of the Western blotting showing a CD80 activity.

FIG. 2A shows the result of RT-PCR in an exemplary cell line expressing CD80 according to the present invention, and FIG. 2B shows a flow cytometry analysis of the same cell line.

FIG. 3 shows a flow cytometry analysis showing a phenotype of DCs.

FIG. 4 shows a flow cytometry analysis showing a phenotype of a cell line expressing CD80 according to the present invention.

FIG. 5 shows a flow cytometry analysis measuring the induction of EBV antigen-specific CTLs using a cell line expressing CD80 according to the present invention.

FIG. 6 shows a flow cytometry analysis measuring the induction of Mart-1 antigen-specific CTLs according to an exemplary cell line expressing CD80 according to the present invention.

FIG. 7 shows a flow cytometry analysis measuring the induction of EBV antigen-specific CTLs using DC.

FIG. 8 shows a flow cytometry analysis measuring the induction of Mart-1 antigen-specific CTLs using DC.

FIG. 9 shows a flow cytometry analysis measuring the induction of EBV antigen-specific CTLs using a cell line expressing CD80 and IL-2 according to the present invention.

FIG. 10A shows a flow cytometry analysis in another cell line expressing CD80 according to the present-invention, and FIG. 10B illustrates the result of RT-PCR of the same cell line.

FIG. 11 shows the result of RT-PCR in a cell line expressing CD86 according to the present invention.

FIG. 12 shows a flow cytometry analysis measuring the induction of EBV antigen-specific CTLs using another cell line expressing CD86 according to the present invention.

FIG. 13 shows a flow cytometry analysis measuring the induction of Mart-1 antigen-specific CTLs according to another cell line expressing CD86 according to the present invention.

FIG. 14A shows the result of RT-PCR in yet another cell line expressing CD80 according to the present invention, and FIG. 14B shows a flow cytometry analysis in another established cell line stably expressing CD80 according to the present invention.

FIG. 15A shows the result of RT-PCR in yet another cell line expressing CD86 according to the present invention, and FIG. 15B shows a flow cytometry analysis in another established cell line stably expressing CD86 according to the present invention.

### DESCRIPTION OF THE PREFERRED EMBODIMENTS

The following is a description of certain embodiments of the present invention.

The method for inducing CTLs according to the present invention (in the following, referred to as “the inducing method of the present invention”) includes bringing a cell line that expresses at least MHC class I and a co-stimulatory molecule into contact with a disease antigenic peptide and further co-culturing the cell line with lymphocytes.

Herein, the “disease” is not particularly limited but is, for example, cancer or infectious disease. There is no particular limitation on the cancer, and any cancer can be addressed The infectious disease is not particularly limited but can be, for example, AIDS, viral infections such as hepatitis B and C, cellular infections, bacteria, mycoses or protozoiases.

After or while bringing the cell line expressing at least MHC class I and a co-stimulatory molecule into contact with a disease antigenic peptide, this cell line is contacted or co-cultured with lymphocytes, so that a disease antigen-specific CTL can be induced. Here, “bringing into contact” means combining the disease antigenic peptide with a MHC class I molecule. More specifically, for example, the cell line is cultured in a culture solution in which the disease antigenic peptides are dissolved, thereby combining the disease antigenic peptides with the MHC class I molecules.

In order to induce CTLs, it is necessary that a complex of an antigenic peptide and MHC class I antigen is recognized by a T cell receptor, a co-stimulatory molecule binds to a CD28 molecule expressed on the T cell surface, and then the T cell is activated. Conventionally, mainly APCs are considered to have an ability to induce CD8-positive CTLs from an antigen presentation by MHC class I molecules and co-stimulatory molecules. However, the inventors of the present invention found for the first time that, using cells other than APCs that do not even express MHC class II molecules, antigen-specific CD8-positive CTLs can be induced by the antigen presentation by MHC class I antigen and expression of the co-stimulatory molecules.

In other words, in the conventional method for inducing CTLs it has been necessary to use patient's own cancer cells as APCs to present antigen for every induction, so that the processes are very complicated. In contrast, according to the method of the present invention, since the cell lines can be utilized as the APCs, it becomes possible to exclude the induction processes considerably and reduce burdens on patients.

In the case of HIV infection or AIDS, the HIV virus also infects APCs such as DCs and CD4-positive T cells necessary for induction of CD8-positive T cells (namely, CTLs). Therefore, when carrying out a CTL therapy according to the conventional method, for example, the obtained CTLs also are infected with HIV and cannot be used for treatment. However, since the inducing method of the present invention does not need to use such DCs, it becomes possible to induce uninfected CTLs (CD8-positive T cells), allowing a CTL therapy for HIV patients that was not possible in the past.

Also, not allowing antigenic peptides to be expressed at a cell line that transmits a stimulus for inducing CTLs but bringing the antigenic peptides into contact with the cell line individually makes it possible to induce CTLs specific for various diseases even with only one cell line simply by changing the kinds of the antigenic peptides. Consequently, in the inducing method of the present invention, various diseases can be addressed immediately.

Furthermore, in the conventional method for inducing CTLs, when inducing CTLs for multiple diseases, it has been necessary to utilize cells expressing respective antigens. In contrast, in the inducing method of the present invention, there is no need for establishing multiple cell lines, so that CTLs can be induced in a simple manner. In addition, since more CTLs can be obtained compared with the conventional case, the induction efficiency of CTLs also improves.

The following description is directed to the inducing method of the present invention. A cell line of the present invention will be described. The cell line expressing co-stimulatory molecules used in the inducing method of the present invention (in the following, referred to as “the cell line of the present invention”) is not particularly limited as long as it expresses MHC class I and the co-stimulatory molecules. For example, an artificially produced cell line or the like can be used. Herein, “express” means that the class I MHC and the co-stimulatory molecules are arranged on the surface of the cell line of the present invention and expressed in an amount sufficient for exhibiting a CTL induction ability. In addition, the “cell line” refers to one or more cells of the cell line in some cases.

As described later, the cell line of the present invention can be produced by a conventionally known method, for example, by transfecting into the cell line expressing MHC class I antigens an expression vector in which the co-stimulatory molecules are cloned. In the cell line of the present invention, it is preferable that the MHC class I and the co-stimulatory molecules are expressed constantly and stably. This is because the reproducibility of the CTL induction improves, so that the inducing method of the present invention can be made simpler and more efficient. Accordingly, in the case of transfecting the expression vector, for example, a stable transfection by being integrated into chromosome is more preferable than a transient transfection.

MHC class I molecules include heavy chain (alpha chain) MHC class I proteins, such as the human class I heavy chains HLA-A, HLA-B, HLA-C, HLA-E, HLA-F and HLA-G. Preferably, HLA-A, HLA-B or HLA-C heavy chains are used, most preferably HLA-A heavy chains are used. These MHC class I heavy chains are preferably expressed in conjunction with β2-microglobin. The genes expressing these MHC class I proteins are known in the art as found in public databases such as IMGT/HLA Sequence Database (http://www.ebi.ac.uk/imgt/hla), for example, the GenBank accession numbers of HLA-A*0201, HLA-A*2402, HLA-A*3302, HLA-A*1101, HLA-A*0101, and HLA-A*0301 are M84379, M64740, L06440, D16841, M24043, and U32184, respectively.

Examples of the co-stimulatory molecules include CD80 (B7.1) and CD86 (B7.2). These molecules can transmit a signal for activating the immune function of lymphocytes. When they recognize their counter ligand CD28 on the surface of lymphocytes, the lymphocytes are activated, thus inducing CTLs.

In the cell line of the present invention, it is appropriate that either CD80 or CD86 is expressed, and preferably CD80 is expressed. Alternatively, both CD80 and CD86 may be expressed simultaneously.

Also, various other molecules may be expressed on the cell surface. Adhesion molecules such as CD54 (ICAM-1), ICAM-2, ICAM-3, or LFA-3, or other co-stimulatory molecules may be expressed. Also, molecules such as CD1b that can presents glycolipids or CD1d presenting α-galactosylceramide for enhancing the induction of NKT cells may be expressed.

Other than the stimulatory molecules, other useful genes may be integrated and expressed. For example, a suicide gene, such as HSV-TK, may be integrated, to make it possible to remove the APC line and collect CTLs alone in an efficient manner by carrying out a predetermined procedure after the CTL induction.

Furthermore, for the cell line of the present invention, any cells can be used preferably as long as the cell line can express the MHC class I and co-stimulatory molecules. Zooblasts are preferable, and human cells are more preferable.

Among these cells, cells derived from tumor are preferable The resultant established cell line can be constantly proliferated on a semipermanent basis and has a stable property although the establishment of the tumor derived cell line is not so easy.

These cell lines are available from various culture collections or can be produced by a conventional method.

The type of MHC class I of the cell line of the present invention may be any type of HLA. As the type of HLA class I, HLA-A2 or HLA-A24 is preferable, HLA-2 homozygote or HLA-A24 homozygote is more preferable, and HLA-A*0201 homozygote or HLA-A*2402 homozygote is particularly preferable.

Examples of such cell lines include breast cancer-derived MDA-MB-231 (hereafter referred to as MDA-MB) (class I antigen HLA-A2), renal cancer-derived TUHR10TKB (class I antigen HLA-A2/24) and stomach cancer-derived JR-St (class I antigen HLA-A24). These cell lines are available through Cell Banks such as ATCC and RIKEN BioResource Center.

These HLA types mentioned above account for about 60% in humans (see homepage of Japanese Society for Histocompatibility and Immunogenetics, HLA Data Library, Gene frequencies of serologically-typed HLA-A, B, C loci: (http://square.unim.ac.jp/JSHI/hla_data/freq/hla_freq.txt)). Since CTLs induced at these cell lines having the above HLA types become HLA-A2-restricted or HLA-A24-restricted, there is an advantage that those CTLs can be used for many patients in not only Japanese donors but also other donors over the world.

Further, another aspect of the inducing method of the present invention includes co-culturing of lymphocytes with a cell line that expresses at least MHC class I, a disease antigen and a co-stimulatory molecule.

Alternatively, lymphocytes may be contacted with disrupted cells or isolated membranes containing the MHC class I molecule(s), loaded with the antigenic-peptide, and which contain co-stimulatory molecules, such as CD80 and/or CD86, and optionally other accessory or adhesion molecules.

The cell line may also be selected from cell lines known to express MHC class I molecules which are “empty”, that is, have peptide binding pockets that have not yet been loaded with an antigenic peptide. For example, mutant cell lines that are defective in the peptide loading process, such as RMA-S or T2, may be employed.

It is also possible to express an exogenous disease antigen in a cell line expressing MHC class I molecules and co-stimulatory molecules, such as CD80 or CD86. This eliminates the need for contacting or co-culturing the cell line with antigenic peptides. Thus, the induction of CTLs can be simplified further.

In the manner described above, in an embodiment of the inducing method of the present invention, a complex of an HLA-A2 molecule and an antigenic peptide on the cell line expressing MHC class I molecules and co-stimulatory molecules is recognized by a T cell receptor, and further, the lymphocyte is activated by co-stimulation of CD80 on the cell line, thus induced to be a CTL. As a result, the CTL responds specifically to a certain cancer antigen, and the percentage of responding to substances other than that antigen lowers compared with a conventional method, leading to increased specificity. This makes it possible to provide CTLs having a high specificity for a disease site, thus allowing a highly effective treatment.

Some exemplary cell lines according to the present invention are:
1) MDA-MB/CD80
2) MDA-MB/CD80, HSV-TK
3) MDA-MB/CD80, HLA-A*0201, β2M
4) MDA-MB/CD80, CD1b
5) MDA-MB/CD80, CD1d
6) MDA-MB/CD80, CD86
7) MDA-MB/CD80, PAP
8) MDA-MB/CD80, AFP
9) MDA-MB/CD80, MUC1
10) MDA-MB/CD86
11) MDA-MB/CD86, PAP
12) MDA-MB/CD86, AFP
13) TUHR10TKB/CD80
14) TUHR10TKB/CD80, PAP
15) TUHR10TKB/CD80, AFP
16) TUHR10TKB/CD80, MUC1
17) TUHR10TKB/CD86
18) JR-St/CD80
19) JR-St/CD86

In the above 3), β2M refers to β2 microglobulin. In the case where MHC class I molecule is presented on the cell surface, the MHC class I heavy chain molecule is known to form a complex with the molecule of β2 microglobulin. The presence of these molecules allows the MHC class I molecules to be expressed in a stable manner. In other words, by allowing β2M to be expressed, MHC class I molecules also can be expressed on the surface stably, making it possible to present the complex with a disease antigenic peptide stably.

PAP, AFP and MUC1 used in the above 7), 8), 9), 11), 12), 14), 15) and 16) indicate a prostate cancer antigen, a hepatoma antigen and a pancreatic cancer antigen, respectively.

The cell line of the present invention can be produced suitably using a conventionally known method. For example, the document below can be referred to.

Anal Biochem. 1993 Feb. 1;208(2):352-6. Maximal expression of Recombinant cDNAs in COS cells for use in expression cloning. Kluxen F W, Lubbert H.

More specifically, the following procedures may be followed, for example.

1) After genes of CD80 or other molecules that are to be expressed on the cell line surface are amplified so as to obtain cDNA, this cDNA is integrated into a suitable expression vector. In the case where a disease antigen such as a cancer antigen also is to be expressed on the cell line, genes thereof also are integrated at the same time.

2) The expression vector is transfected into a cell line to be used, followed by culturing in a culture medium. Then, selection is made in a culture medium containing antibiotic.

3) Furthermore, using antibody combining beads, cells expressing recombinant protein (genes to be expressed) are concentrated.

4) The above-noted cells concentrated by the beads are selected and cloned by a limiting dilution method or the like, and then a stably expressed cell line is obtained. In the case of allowing a disease antigen to be expressed as well, its expression can be confirmed by the Western blotting or the like.

Examples of the expression vectors that can be used in the above-mentioned technique include pRc/CMV, pZeoSV2, pBudCE4-1 and pcDNA/V5-His (manufactured by Invitrogen Corporation).

Examples of the culture medium include Eagle's MEM, Dulbecco's MEM and RPIM1640.

Examples of the antibiotic include neomycin, Zeocin, Blasticidin-S and Hygromycin.

The disease antigen and the disease antigenic peptide used in the present invention (in the following, referred to as “the peptide of the present invention”) can be, for example, antigen and antigenic peptide associated with various diseases described above. By using a cancer antigen, an infectious disease antigen and peptides thereof, for example, a precise treatment can be conducted. A peptide sequence that can be used as the peptide of the present invention can be a known antigenic peptide sequence depending on the kinds of cancers and infectious diseases and HLA types indicating restriction properties. Further, the above-noted peptide sequence are able to be identified by a conventionally known method, for example, screening using a computer algorithm or an immunologic technique (see Kuzusima et al., BLOOD, 2003, vol. 101, no. 4, 1460-1468, for example).

Peptides which bind to MHC class I molecules and which are associated with infectious diseases or cancers are described by Romero et al (J Exp Med, 188:1641-1650, 1998), Khanna et al (Proc Natl Acad Sci USA 96 1039-10396, 1999), Oka Y et al (Immunogenetics. 51(2):99-107, 2000), Butterfield L H et al (J. Immunol. 166(8):5300-8, 2001), Ferrara A et al (J Cancer Res Clin Oncol. 129(9):521-30 2003), Tan L C et al (Arthritis Res. 2(2):154-64 2000), Kawakami Y et al (J. Immunol. 154(8):3961-8 1995), van der Bruggen P et al (Eur J. Immunol. 24(12):3038-43 1994), Kawakami Y et al (J Exp Med. 180(1):347-52 1994), Hiltbold E M et al (Cancer Res. 58(22):5066-70 1998), Brossart P et al (Blood. 93(12):4309-17 1999), Domenech N et al (J. Immunol. 155(10):4766-74 1995), JP 2003-535024 A, WO0006723A1, Parkhurst M R et al (Cancer Res. 58(21):4895-901 1998), Robbins P F et al (J. Immunol. 159(1):303-8 1997), Fujie T et al (Int J Cancer. 80(2):169-72 1999), Tanaka F et al (Cancer Res. 57(20):4465-8 1997), Shichijo S et al (J Exp Med. 187(3):277-88 1998), Wang R F et al (J. Immunol. 161(7):3598-606 1998), Wang R F et al. (J Exp Med. 183(3):1131-40 1996), Wang R F et al (J Exp Med. 184(6):2207-16 1996), Tissue Antigens (Immunex Corperation, Seattle, Wash., USA. 60(1):16-24 2002), Brichard V G et al (Eur J. Immunol. 26(1):224-30 1996), Pold M et al (Genomics. 15;59(2):161-7 1999), and Pierre G et al (Annu. Rev. Immunol. 20:621-667 2002), which are hereby incorporated by reference. Table 1 below describes examples of the cancer antigenic peptides of the present invention.

TABLE 1

| | | cancer |
|---|---|---|
| HLA-A0201 restricted antigenic peptides | | |
| WT-1 | RMFPNAPYL | various kinds of cancers |
| AFP-1(325-334) | GLSPNLNRFL | lung cancer |
| AFP-2(158-166) | FMNKFIYEI | lung cancer |
| AFP-3(137-145) | PLFQVPEPV | lung cancer |
| AFP-4(542-550) | GVALQTMKQ | lung cancer |
| AFP-4-1(modified)-A.2.1 | GVALQTMKL | lung cancer |
| HPV18E7(86-94) | FQQLFLNTL | cancer of the uterine cervix |
| HLA-A2 restricted antigenic peptides | | |
| BMLF1(derivd from EBV) | GLCTLVAML | B-cell lymphoma |
| gp100 | KTWGQYWQV | melanoma |
| Humman papilloma virus | LLMGTLGIV | cancer of the uterine cervix |
| type 16E7 | | |
| MAGE-3 (JH7363) | FLWGPRALV | melanoma, various cancers |
| MART-1 | AAGIGILTV | melanoma |
| MART-1 modified (A27L) | ELAGIGILTV | melanoma |
| MUC1 | PDTRPAPGSTAPPAHGVTSA | pancreatic cancer, others |
| MUC-1.1 modified | STAPPVHNV | pancreatic cancer, others |
| MUC-1.1 naive | STAPPAHGV | pancreatic cancer, others |
| MUC-1.2 | LLLLTVLTV | pancreatic cancer, others |
| PAP-10 | PLERFAELV | prostate cancer |
| PAP-5 | ALDVYNGLL | prostate cancer |
| TRP2-1 | SVYDFFVWL | melanoma |
| HLA-A24 restricted antigenic peptides | | |
| gp100 | VYFFLPDHL | melanoma |
| MAGE-1 | NYKHCFPEI | melanoma, various cancers |
| MAGE-3 | IMPKAGLLI | melanoma, various cancers |
| HLA-A26 restricted antigenic peptides | | |
| SART-1 | KGSGKMKTE | various kinds of cancers |
| HLA-A31 restricted antigenic peptides | | |
| NY-ESO-1 | LAAQERRVPR | melanoma, various cancers |
| TRP1 | MSLQRQFLR | melanoma |
| TRP2-2 | LLGPGRPYR | melanoma |
| HLA-B44 restricted antigenic peptides | | |
| MAGE-3 | MEVDPIGHLY | melanoma, various cancers |
| Tyrosinase | SEIWRDIDF | melanoma |
| HLA-DRB7 restricted antigenic peptides | | |
| ela2 peptide | EGAFHGDAEALQRPVAS | acute lymphoblastic |
| | | leukemia |

There is no limitation on the kinds of cancer antigens which may be 5 used so long as the antigenic determinants of these antigens are presentable by MHC class I molecules. For example, as illustrated above, antigen of any cancer including a prostate cancer, a hepatoma and a pancreatic cancer can be utilized. Tumor specific antigens include those encoded by the MAGE gene family, such as MAGE 1 and MAGE 3, GAGE, BAGE and RAGE. Other cancer-associated antigens arising from mutations, such as p53, K-ras, CDK4 and the bcl-c-abl gene product, antigens over-expressed in cancer cells, such as c-erb2 (or neu) protein; as well as oncogenic viral antigens, such as the E7 protein of HPV-16 are included. Oncofetal antigens, such as CEA (carcinoma embryonic antigen) or alpha-fetoprotein (AFP), as well as differentiation antigens, such as prostate specific antigen and CD-10 (CALLA antigen), which is expressed in B-cell leukemias and lymphomas, may also be used.

There also is no limitation on the kinds of infectious diseases. For example, antigens of diseases that are intractable among other viral infections, for example, HIV (AIDS), hepatitis A/B/C, Epstein-Barr Virus (EBV), and HPV infection can be used to induce CTLs that are effective against these diseases and to carry out better treatment, which has been difficult conventionally. Antigens from parasites such as Plasmodium circumsporozoite protein may also be employed.

Compared with a conventional method for inducing CTLs using cancer antigen harvested from patient's own cancer tissues, the present invention uses a synthetic peptide and therefore imposes less burden on the patient.

Lymphocytes used in the present invention can be of various kinds but preferably are human lymphocytes. It is preferable to use patient's own lymphocytes or allogenic lymphocytes which share with MHC class I molecules on the cell line of the present invention because CTLs particularly specific for individual patients can be induced and thus are not rejected owing to the recognition of allogeneic antigen.

Here, the “own lymphocytes” refer to lymphocytes harvested from a patient. Such a patient may be a patient who has already developed cancer or an infectious disease or a patient at risk of developing cancer or an infectious disease, for example, a patient who is in a precancer state or is a carrier of an infectious disease.

As a culturing method for inducing CTLs, it is possible to use various known methods associated with the culture of human cells, in particular, lymphocytes. For example, the methods described in the documents below preferably are used for culturing.

Blood, 91; 977-983, 1998; Dendritic cells stimulate the expansion of bcr-abl specific CD8+ T cells with cytotoxic activity against leukemic cells from patients with chronic myeloid leukemia, M. Nieda et al.

Eur J. Immunol. 1980 January; 10(1): 30-5. Generation of virus-specific cytotoxic cells in vitro. II. Induction requirements with functionally inactivated virus preparations, Koszinowski U H, Gething M J.

J. Immunol. 1988 Nov. 1; 141(9): 2975-9. A role of HLA-DQ molecules of stimulator-adherent cells in the regulation of human autologous mixed lymphocyte reaction, M. Nieda et al.

The condition is appropriate for culture as long as it satisfies the ranges below. In 1 ml-culture solution, for example, the concentration of reactive cells (lymphocytes) is from 1×10⁶ to 2×10⁶ cells/ml, that of the cell lines of the present invention is from 1×10⁵ to 5×10⁵ cells/ml, and that of peptides is from 1 to 10 μg/ml.

In addition, although the culture medium preferably is an AIM-V medium or a RPMI 1640 medium to which 1% to 10% FCS or AB serum or patient's own plasma is added.

The procedure of an inducing method as an example can be as follows.

1) The cell line expressing CD80 according to the present invention is brought into contact with an antigenic peptide so as to be sensitized, and then treated with antibiotic such as mitomycin or irradiated.

2) The above cell line is suspended in a culture medium, and lymphocytes or a cell fractionation containing lymphocytes are added to this cell line, followed by culturing for 6 to 8 days.

3) Thereafter, the antigenic peptide is added appropriate times as necessary, and stimulated and cultured until desired CTLs are obtained.

Now, CTLs induced by the inducing method of the present invention (in the following, referred to as “the CTLs of the present invention”) will be described.

The CTLs induced by the inducing method of the present invention are more likely to function specifically to individual disease antigens than CTLs induced conventionally as described above and therefore can be used for a cell-mediated immunotherapy, for example, which has an excellent therapeutic effectiveness.

Also, simply by using an antigenic peptide of an intended disease antigen for co-culturing, it becomes possible to induce CTLs specific to various disease antigens, thereby providing these CTLs for a wide range of treatments.

Containing the CTL as a principal ingredient, a drug of the present invention can be formulated as a therapeutic agent for diseases including intractable diseases, for example.

Concerning the construction of the drug, besides the CTL, it also is possible to combine cytokine effective for cancer treatment such as IL-2 or IL-12 in the case of cancer treatment or IFN (interferon)-γ or α in the case of treating viral infections such as hepatitis with the CTL. Also, a molecular target drug can be combined.

The CTLs of the present invention induced specifically to a certain cancer can be administered as an agent treating for cancer instead of a conventional antitumor agent, thereby achieving a highly effective therapy with virtually no side effects.

Further, viral infections such as AIDS and hepatitis B/C are difficult to treat at present, and a conventional treating method may cause side effects and achieves only a low effectiveness. However, CTLs induced specifically to these diseases make it possible to provide highly effective manner as a sort of drug for solving the above-mentioned problems.

Further, with such a construction, the drug of the present invention can function as a so-called prophylactic agent against cancer, for example, for preventing metastasis or recurrence after operation or for treating in a precancer state. For viral infections, the drug of the present invention can prevent onset of the disease even in a so-called silent carrier phase before the onset.

The following is a description of a treating and preventing method using the CTLs of the present invention. The treating and preventing method of the present invention is characterized by using the CTLs of the present invention.

For example, not only can the CTLs be used as a principal component in the drug as described above, but also the induced CTLs can be administered directly. When administering, it is possible to infuse the CTLs into a lesion of cancer directly or by an intravenous injection. Alternatively, they can be administered by not only an intravenous injection but also an intraderaml injection or direct injection in the draining lymph node. Alternatively, it also may be possible to infuse these CTLs from an artery in the vicinity of the lesion. When the CTLs are contained as a principal component, the agent used in combination therewith can be, for example, LAK cells, NK cells, NKT cells, γδT cells or cytokine.

In any cases, the function of infused CTLs and the immune strength improved by these CTLs allow a highly effective treatment.

A kit for inducing CTLs according to the present invention is characterized by including a disease antigenic peptide and a cell line that expresses MHC class I and a co-stimulatory molecule. According to a conventional inducing method, it has been impossible to provide a kit in a simplified manner because antigen-presenting cells and cells expressing CD80 have to be produced every time CTLs are induced and cancer cells need to be harvested from a patient. However, the present invention has made it possible to provide a kit using, for example, a CD80-expressing cell line and an antigenic peptide. Accordingly, CTLs can be induced in a simplified and precise manner.

Further, the above-noted kit also can include suitably a culture medium, a reagent, etc. if necessary.

As another aspect of the above-described kit for inducing CTLs, a kit including a cell line that expresses MHC class I, a disease antigen and a co-stimulatory molecule may be provided. In the case of addressing a specific disease rather than various diseases, when the kit including a cell line that expresses antigen of this disease is provided, it becomes unnecessary to bring the antigenic peptide into contact again, thus achieving a further simplified CTL induction.

A method for testing a proliferation potential of CTLs according to the present invention is characterized by co-culturing a lymphocyte harvested from a subject while bringing a disease antigen and a cell line expressing a co-stimulatory molecule into contact with each other, thereby determining an ability to induce CTLs.

Although the effectiveness of the CTL therapy has been already made clear compared with other cell-mediated immunotherapies, CTLs cannot necessarily be induced from any patients depending on the properties of lymphocytes. Accordingly, the test method of the present invention can be used for determining before starting the treatment whether the CTL therapy is possible.

After a slight amount of lymphocytes is harvested as a sample from a patient, whether or not CTLs can be induced is determined by the test method of the present invention. If it is confirmed, then as an actual treatment, therapeutic CTLs can be induced using a known method or the inducing method of the present invention. This also makes it possible to determine the effectiveness of an immunotherapy to be conducted later.

As another aspect of a method for testing a proliferation potential of CTLs according to the present invention, it may be possible to provide a test method of co-culturing of lymphocytes harvested from a subject with a cell line that expresses a disease antigen presented by MHC class I molecules and expresses a co-stimulatory molecule, thereby determining an ability to induce CTLs. In the case of addressing a specific disease rather than various diseases, when the test method using a cell line presenting an antigen specific for a disease (a disease antigen) is provided, it becomes unnecessary to bring the antigenic peptide into contact again, thus achieving a further simplified CTL induction.

A conventional test method has been inconvenient in that a testing needs to be conducted after harvesting tissues from the subject or preparing DCs. However, according to the present invention, it is appropriate to collect only a slight amount of PB, so that a burden on the subject is alleviated considerably.

As described above, by using the method for inducing CTLs according to the present invention, the CTLs induced by this inducing method and uses thereof such as a drug or a treating method, it becomes possible to provide treatments that are more effective than a conventional treatment, in particular, treatments of cancer and viral infections.

Furthermore, in the case of cancer treatment, they also can be used effectively for preventing metastasis or recurrence after operation, thereby improving a curing ratio and a life-prolonging effect. In the case of viral infections, it becomes possible to prevent recurrence.

The following is a specific description of the present invention by way of examples. It is needless to say that the present invention is not limited to these examples.

### EXAMPLE 1

<Establishment of a Cell Line Stably Expressing CD80: MDA-MB/CD80>

(1) Cloning of Gene Fragment

A cDNA library was synthesized from total RNA fraction obtained from CD80 expressing cells by using reverse transcriptase. Then the CD80 cDNA fragment was amplified by PCR method. As a primer set, two kinds of synthetic DNA oligomers listed below were used. The underlined sequences in the primer sequences below respectively indicate the sequences Hind III (CD80: F) and Xba I (hB7-1: R) of restriction sites to be inserted into a vector.

| | |
|---|---|
| Primer CD80: F | |
| 5′-TTCAAGCTTACCATGGGCCACACACGGAGGCA | (SEQ ID NO: 1) |
| GGGAACATCACC-3′ | |
| Primer hB7-1: R | |
| 5′-TAATCTAGATGCGGACACTGTTATACAGG-3′ | (SEQ ID NO: 2) |

The PCR method was carried out under the following condition of reaction. The reaction mixture containing LATaq DNA polymerase (manufactured by Takara) was first allowed to stand at 94° C. for 3 minutes, then subjected to 30 cycles each of which consisted of 94° C. for 30 seconds, 55° C. for 30 seconds and 72° C. for 30 seconds, and finally allowed to react at 72° C. for 5 minutes. The DNA fragment specifically amplified by the above-described PCR method was isolated after 1.5% agarose gel electrophoresis, treated with restriction enzymes Hind III and Xba I, and inserted between Hind III and Xba I sites of plasmid pRc/CMV (manufactured by Invitrogen Corporation), thus obtaining pRc/CMV-CD80. When the inserted sequence in the pRc/CMV-CD80 was analyzed by a DNA sequencer, it perfectly matched with the sequence of human CD80 cDNA reported in the paper [Freeman G. J. et al. (J. Immunol.) 143 2714-2722 (1989)].

(2) Measurement of CD80 Activity

CD80 directly binds to CD28, thereby transmitting a signal into a cell expressing CD28. Accordingly, the activity of CD80 cloned in the above-noted pRc/CMV-CD80 was measured by analyzing phosphorylation of CD28 in Jurkat cells expressing CD28.

Using a laboratory dish with a diameter of 35 mm, CHO cells were cultured in RPMI 1640 medium containing 10% fetal bovine serum for 4 to 6 hours. Into these cells, 6 μg of the pRc/CMV-CD80 was transfected by a lipofection method, thus obtaining CHO-CD80 cells. Also, pRc/CMV that does not code CD80 gene was introduced similarly into CHO cells, thus obtaining CHO-mock cells.

5×10⁶ cells of the CHO-CD80 or CHO-mock cells cultured in the RPMI 1640 medium containing 10% fetal bovine serum and 1×10⁷ cells of the Jurkat cells cultured in the RPMI 1640 medium containing 10% fetal bovine serum respectively were transferred into RPMI 1640 medium containing no 10% fetal bovine serum, mixed in 1.5 ml tubes and maintained at 37° C. for 10 minutes. Thereafter, an equivalent amounts of 2× cytolytic buffer solution (20 mM Tris-HCl (pH 7.5), 137 mM NaCl, 1 mM MgCl₂, 1 mM CaCl₂, 10% glycerol, 1% Nonidet P-40, 150 μM Na₃VO₄, 20 μg aprotinin, 200 μM PMSF) was added to lyse these cells, and then the cell extract was subjected to centrifugal separation (14000 rpm, 4° C., 15 minutes), thereby obtaining a supernatant.

Then, 50 μl of 50% suspended protein G Sepharose that was equilibrated with 1× cytolytic buffer solution was added to each of the above supernatant and stirred at 4° C. for 1 hour, followed by centrifugal separation (3000 rpm, 4° C., 1 minute). Subsequently, 15 μl of 200 ng/μl anti-CD28 antibody was added to the supernatant separated by the centrifugal separation and stirred at 4° C. for 1 hour. Thereafter, 20 μl of 50% suspended protein G Sepharose was added and stirred at 4° C. for 1 hour, followed by centrifugal separation (3000 rpm, 4° C., 1 minute). The obtained precipitate was washed five times by 900 μl of 1× cytolytic buffer solution, and then 2× SDS loading dye in an amount equal to the remaining solution was added, thus producing a sample of SDS gel electrophoresis.

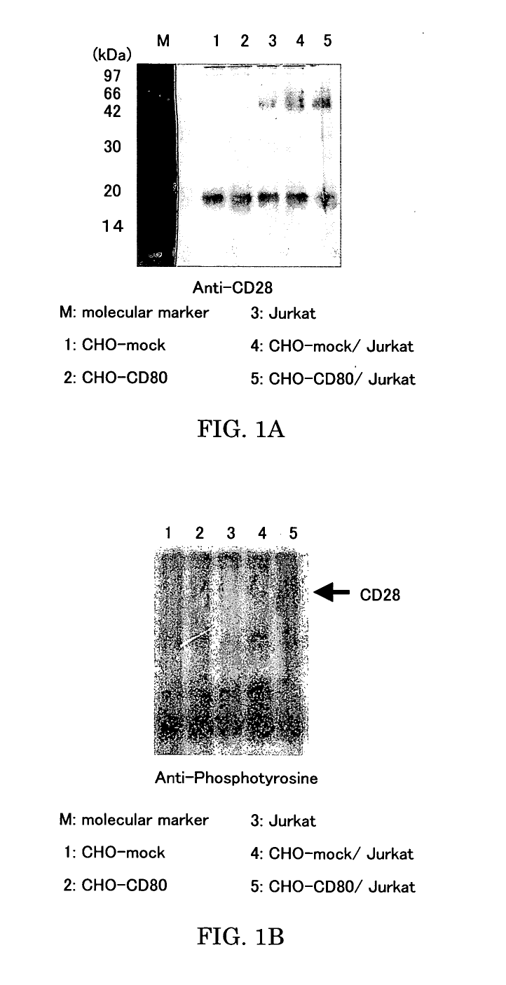

The Western blotting for detecting phosphorylation of CD28 was carried out using anti-phosphorylation tyrosine antibody by separating the sample obtained above by 12% SDS gel electrophoresis (SDS-PAGE) and then transferring the proteins into a PVDF membrane. FIGS. 1A and 1B show an example of the result. FIG. 1A shows a gel stained after SDS-PAGE, while FIG. 1B shows the result of the Western blotting. In these figures, lane 1 indicates the case of using CHO-mock alone, lane 2 indicates the case of using CHO-CD80 alone, lane 3 indicates the case of using Jurkat alone, lane 4 indicates the case of using CHO-mock and Jurkat, and lane 5 indicates the case of using CHO-CD80 and Jurkat. As shown in lane 5 of FIG. 1B, CD80 cloned in the above-noted pRc/CMV-CD80 was able to phosphorylate CD28 and thus was shown to have an activity as co-stimulatory molecules.

(3) Establishment of cell Line Stably Expressing CD80

A cell line MDA-MB/CD80 expressing human CD80 stably and constantly in MDA-MB-231 cells was established as follows.

Using a laboratory dish with a diameter of 35 mm, MDA-MB-231 cells were cultured in RPMI 1640 medium containing 10% fetal bovine serum for 4 to 6 hours. Into these cells, 6 μg of the pRc/CMV-CD80 was transfected by a lipofection method. 48 hours after the transfection, a transient expression of CD80 was confirmed by a flow cytometry. At the same time, the above-noted medium was replaced with a medium containing antibiotic G418, thus selecting cells with chromosome into which the plasmid was integrated. Furthermore, a cell line was cloned from a single cell of the selected cells by a limiting dilution method, thus obtaining a cell line MDA-MB/CD80_{G418} expressing human CD80 stably.

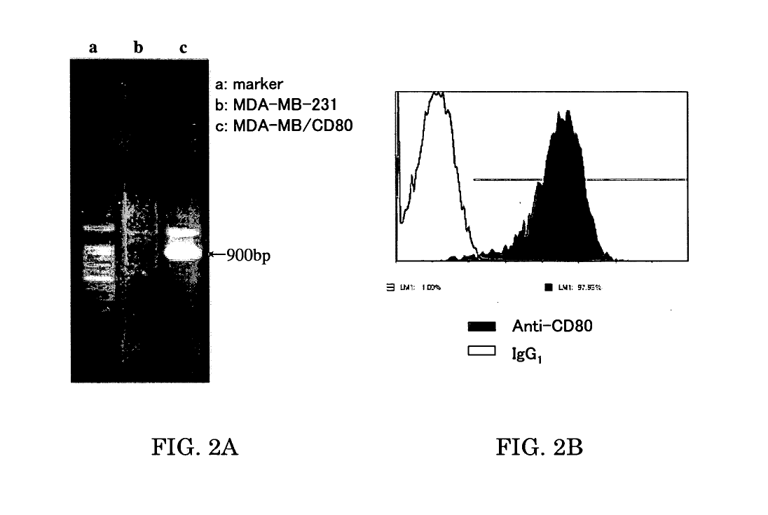

It was confirmed by a RT-PCR method using mRNA extracted from the MDA-MB/CD80_{G418} and a flow cytometry using anti-CD80 antibody that the MDA-MB/CD80_{G418} expressed CD80. FIGS. 2A and 2B respectively show examples of these results. As shown in FIG. 2A, the MDA-MB/CD80_{G418} (lane c) had a higher level of mRNA expression of CD80 than MDA-MB-231 (lane b), which was a parent cell line thereof. Also, as shown in FIG. 2B, it was confirmed by a flow cytometry that CD80 molecules were expressed on the surface of the MDA-MB/CD80.

(4) Phenotype Comparison Between CD80-Expressing Cell Line and Normally Used DC

Next, markers expressed on the surfaces of cell membranes of the CD80-expressing cell line (MDA-MB/CD80_{G418}) and DC were analyzed, thus showing that the method for inducing CTLs by the MDA-MB/CD80_{G418} was different from a conventional inducing method using DC.

(i) Preparation of MDA-MB/CD80 and Measurement of Markers

A sample obtained by suspending 3×10⁵ cells of the MDA-MB/CD80_{G418} described above in 100 μl PBS medium was taken into a Facs tube, stained with antibodies against various markers labeled with FITC (fluorescein isothiocyanate) or PE (phycoerythirin) and then measured by a flow cytometry using COULTER EPICS XL-MCL (measuring device, manufactured by Beckman Coulter, Inc.). The condition of culturing the MDA-MB/CD80 was as follows.
Culture container: Falcon 24-well plate
Used culture medium: AIM-V medium (10% FCS added)
   Note that the above-mentioned medium also may be a RPMI medium or another AIM-V medium using AB serum instead of FCS. In addition, the serum concentration is not limited to 10%. Especially when using AIM-V, no serum is needed.
Culture temperature: 37° C. (5% CO₂)
Culture apparatus: SANYO CO₂ incubator

(ii) Preparation of DC and Measurement of Markers

Monocytes or adherent cell fractinations were cultured in the presence of GM-CSF (500 U/ml) and IL-4 (500 U/ml) for 5 to 8 days, thereby obtaining desired DCs. The condition of culturing DCs was similar to that of culturing the MDA-MB/CD80 described above. A sample obtained by suspending 3×10⁵ cells of the DCs cultured as above in 100 μl PBS medium was taken into a Facs tube, stained with antibodies against various markers labeled with FITC or PE and then measured by a flow cytometry using COULTER EPICS XL-MCL. Further, for comparison, a marker on the surface of MDA-MB-231 cell not expressing CD80 also was measured in a manner similar to the above.

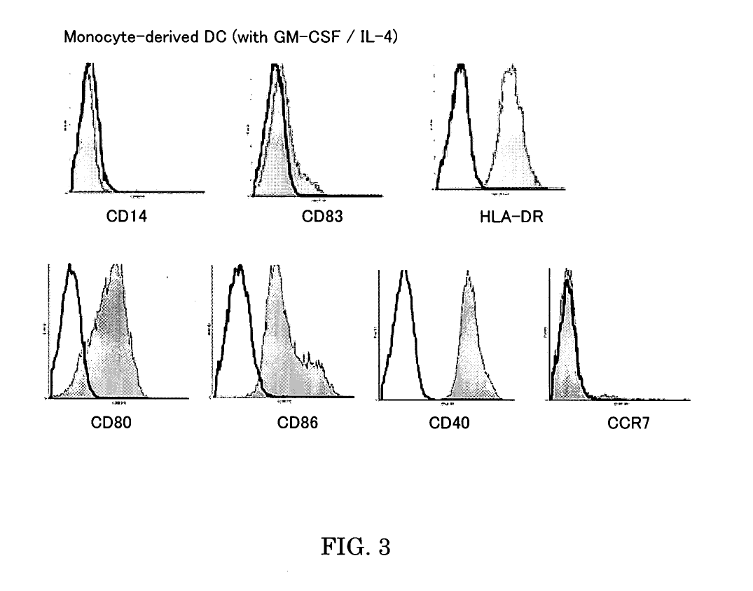

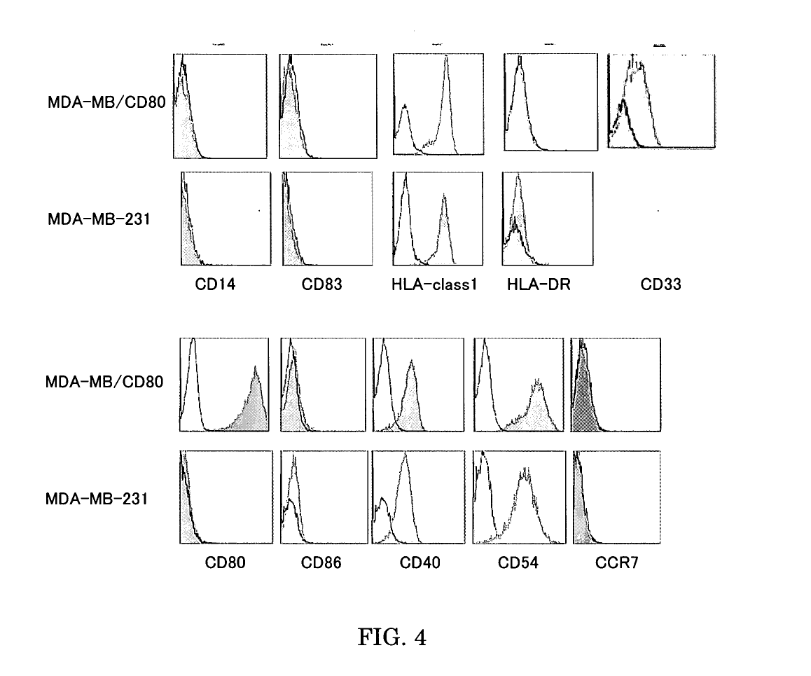

In the following, FIGS. 3 and 4 show the results of the above-described measurements, and Table 2 below describes these results. FIG. 3 shows surface phenotype of DCs induced by a conventional method, while FIG. 4

The markers shown in these figures are listed below.
CD14: marker for checking DC induction (not expressed in DCs)
CD83: marker for DC similar to the above, which becomes positive in the case of mature DC
HLA-DR: marker for Class II antigen
CD80, CD86: co-stimulatory molecules for T cells
CD40: co-stimulatory molecules for T cells
CD33: myeloid cell marker
CD54: adhesion molecules

CCR7: chemokine receptor (expressed on mature DC)

TABLE 2

| | MDA-MB/CD80 | MDA-MB | DC |
|---|---|---|---|
| CD14 | C | C | C |
| CD83 | C | C | B |
| HLA-class 1 | A | A | A* |
| HLA-DR | C | C | A |
| CD80 | A | C | A |
| CD86 | B | B | A |
| CD40 | A | A | A |
| CD33 | A | A* | A* |
| CD54 | A | A | A* |
| CCR7 | C | C | C |

(A: expressed,
B: slightly expressed,
C: not expressed,
*data not shown)

As shown in FIG. 4 and Table 2 above, it was confirmed that the MDA-MB/CD80_{G418} is a myeloid cell belonging to the group of cells including monocytes and DCs, considering that CD33 was expressed. However, the MDA-MB/CD80_{G418} is neither monocytes considering that CD14 was negative nor DCs considering that DR antigen, which was one of MHC class II molecules, was not expressed.

Moreover, considering that MHC class II molecules, which are essential molecules for antigen-presentation by APCs such as monocytes, DCs and B cells this CD80-expressing cell line: MDA-MB/CD80_{G418} did not belong to the group of known antigen-presenting cells. The other characteristics of the surface antigens of this CD80-expressing cell line were the expression of a HLA-class I antigen, the expressions of CD40 and CD54, no expression of CD83 and a slight expression of CD86.

As shown in FIG. 4, CD80, which was not expressed in the parent cell line: MDA-MB-231, was highly expressed on this CD80-expressing cell line. In this manner, the method for inducing CTLs according to the present application can activate and proliferate CD8-positive T cells by allowing an expression of CD80, even though MHC class II molecules, which are essential molecules for antigen-presentation, are not expressed. Furthermore, this cell line originally expresses CD40 and CD54 as well. Accordingly, CD54 molecules increase the contact between lymphocytes and this cell line, and co-stimulatory signals from CD40 further activate and proliferate CD8-positive T cells.

### EXAMPLE 2

<Induction of EBV Antigen-Specific CTL Using MDA-MB/CD80>

The CD80-expressing cells of the MDA-MB/CD80_{G418} were co-cultured with lymphocytes and an Epstein-Barr virus (in the following, abbreviated as EBV) antigenic peptide, which was cancer antigenic peptide, thus confirming an induction of CTLs specific for the cancer antigen. For the above-noted EBV antigenic peptide, a peptide having the following amino acid sequence was used. Further, as a control experiment, a similar procedure was followed for the MDA-MB-231 cells, which were parent cell lines of the MDA-MB/CD80 and did not express CD80.

EBV antigenic peptide (SEQ ID NO: 3): GLCTLVAML

The condition of culturing these cells was as follows.
Temperature: 37° C. (5% CO₂)
Used container: Falcon 24-well plate
Apparatus: CO₂ incubator
Medium: AIM-V medium containing FCS or AB serum, or RPMI 1640 medium

(1) Induction of CTL

After 5×10⁵ cells of MDA-MB/CD80_{G418} were sensitized with the above-noted EBV antigenic peptide (with a concentration of 2 to 10 μg/ml), they were treated with Mitomycin C (manufactured by KYOWA HAKKO KOGYO Co., Ltd.), and then excess Mitomycin C was removed by washing.

The obtained MDA-MB/CD80_{G418} cells were suspended in a 1 ml culture solution, added to each well using 24-well plate, into which 2×10⁶ cells of HLA-A2 (HLA-A*0201) antigen positive T cells or cell fractionation including these T cells were added further, followed by culturing for 6 to 8 days (first stimulation). At this time, in order to enhance an additional antigen-presenting ability, the above-noted peptide (2 to 10 μg/ml) was added to the culture solution.

After culturing, the ratio of the CD8-positive antigen-specific CTLs in the T cells was measured using the EBV antigen-specific tetramer (T-Select MHC Tetramer: manufactured by Medical & Biological Laboratories Co., Ltd.) (measurement after the first stimulation). The measuring method will be described later. Furthermore, a cell population including the CD8-positive antigen-specific CTLs proliferated by the first stimulation was added to the well containing 5×10⁵ MDA-MB/CD80_{G418} cells and the EBV antigenic peptide (2 to 10 μg/ml) that had been prepared as described above, thus providing the second stimulation. After 6 to 8 days of culturing, the ratio of the CD8-positive antigen-specific CTLs in the T cells was measured similarly using the peptide-specific tetramer (measurement after the second stimulation).

The HLA types of the MDA-MB/CD80_{G418} cells used here were as follows.
HLA-A*0201 (type HLA-A2)
HLA-B40/41
HLA-C2/17
HLA-DR14/8

(2) Method for Quantifying CTLs Using Tetramer Specific for EBV Antigenic Peptid

The amounts of inducing the CD8-positive CTLs specific for the EBV antigen after the first stimulation and the second stimulation were analyzed using the tetramer by the following procedure. As the above-noted tetramer, a prepared EBV tetramer (T-Select MHC Tetramer) purchased from MBL (Medical & Biological Laboratories Co., Ltd.) was used.

(I) Binding of Tetramer With the Induced CTL

5 μl of the tetramer and 100 μl of the 5×10⁶ cells/ml CTLs in PBS harvested after the first stimulation and the second stimulation were mixed and incubated at 4° C. for 30 minutes. After washing in PBS and centrifugation for 5 minutes at 4° C. at 1500 rpm, the CTLs were resuspened in 500 μl of PBS and measured by a flow cytometry.

(II) Analysis of CTL Induction Using Tetramer

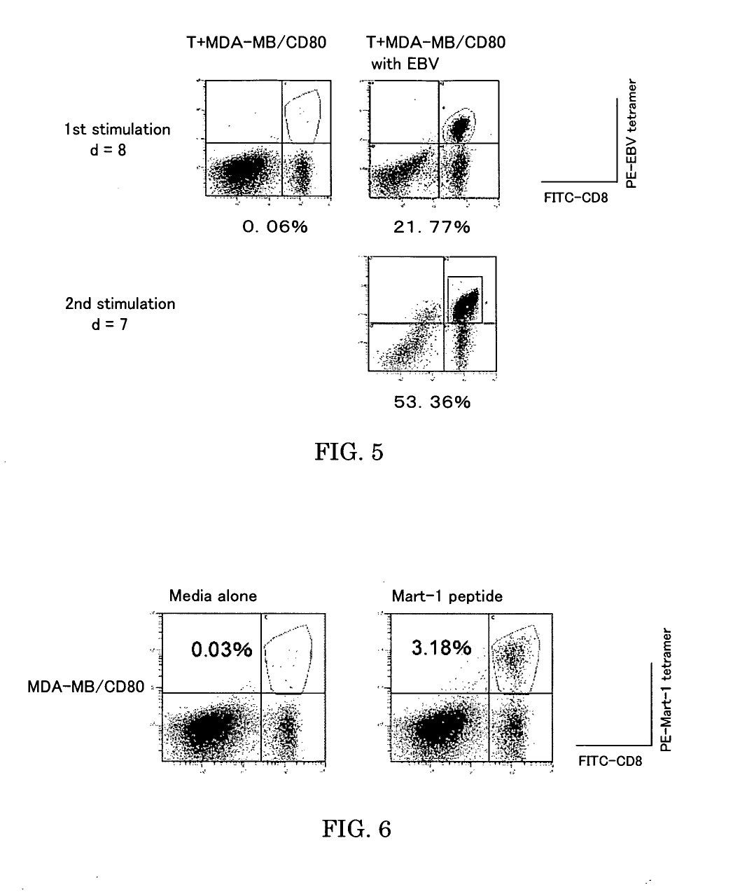

Since the tetramer used here has HLA-A*0201 MHC class I molecules, which form complexes with the above-mentioned MHC-restricted EBV antigenic peptides, it can bind to the CTLs specific for EBV antigen, making it possible to quantify the CTLs precisely. Using the tetramer labeled with PE and the anti-CD8 antibody labeled with FITC, a flow cytometry was conducted so as to measure the induction efficiency of CTLs. FIG. 5 shows an example of the result, and Table 3 below describes the ratio of EBV-specific CTLs.

TABLE 3

| | MDA-MB/CD80 | MDA-MB/CD80 |
|---|---|---|
| | — | EBV-antigen |
| First stimulation | 0.06% | 21.77% |
| Second stimulation | — | 53.36% |

### EXAMPLE 3

<Induction of Melanoma Antigen-Specific CTL Using MDA-MB/CD80>

CD8-positive CTLs specific for a melanoma antigen were induced similarly to Example 2 except for replacing the EBV antigenic peptide with a melanoma peptide (Mart-1), which was a cancer antigenic peptide, and replacing the EBV antigenic peptide-specific tetramer with a Mart-1-specific tetramer (T-Select MHC Tetramer, manufactured by Medical & Biological Laboratories Co., Ltd.). The induction efficiency thereof was measured by a flow cytometry. The Mart-1 peptide was a peptide with the amino acid sequence below. FIG. 6 shows an example of the measurement results, and Table 4 below describes the ratio of Mart-1-specific CTLs.

Mart-1 (SEQ ID NO: 4): ELAGIGILTV

TABLE 4

| | MDA-MB/CD80 | MDA-MB/CD80 |
|---|---|---|
| | — | Mart-1 antigen |
| First stimulation | 0.03% | 3.18% |

### COMPARATIVE EXAMPLE 1

<Induction of EBV Antigen-Specific CTL Using Auto-DC>

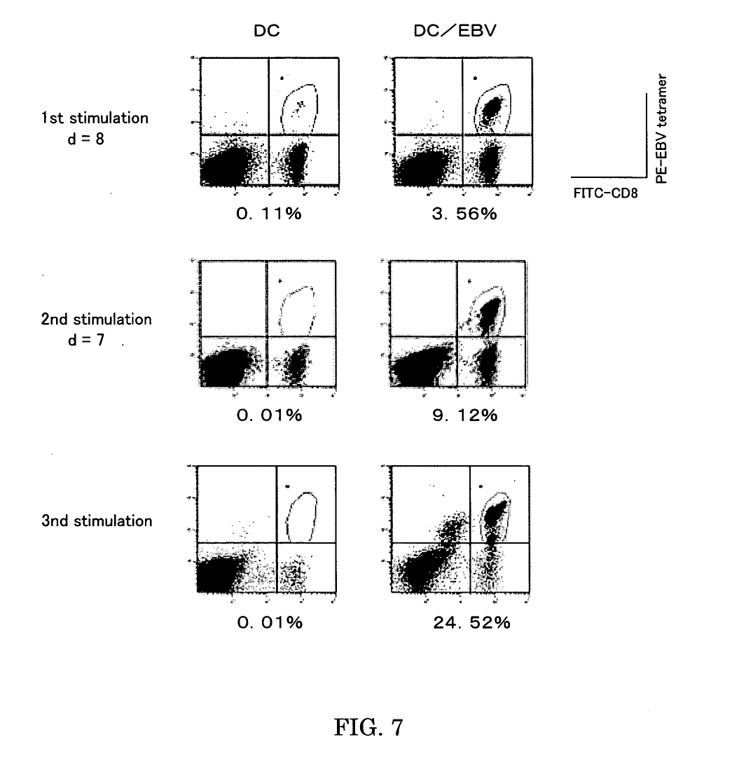

The proliferation ratio of CD8-positive EBV-specific CTLs was measured similarly to Example 2 except the following. The MDA-MB/CD80 was replaced with HLA-A2 (or HLA-A*0201) antigen-positive DC prepared as below. Using this DC as a stimulatory cell and an auto-mononuclear cell fractionation or a cell population containing T cells at a high purity as a reactive cell, auto MLR (auto mixed lymphocyte culture reaction) was carried out in the presence of the EBV antigenic peptide. Then, the second stimulation was provided on 6 to 8 days of culturing, and the third stimulation was provided on 6 to 8 days after the second stimulation, thus collecting part of the cells on 6 to 8 days after each of these stimulations as measurement samples. FIG. 7 shows an example of the measurement results, and Table 5 below describes the ratio of the EBV-specific CTLs.

Preparation of DC

The above-noted DCs were obtained as follows. Using 24-well plate, 6-well plate, 25 ml flask and 50 ml flask etc. according to the number of cells, HLA-A2 (or HLA-A*0201) antigen-positive monocytes derived from peripheral blood or adherent cell fractionations were cultured in the presence of GM-CSF (500 U/ml to 1000 U/ml) IL-4 (400 U to 1000 U/ml) for 5 to 7 days, thus obtaining desired DCs.

The HLA types of the DCs are as follows.
HLA-A*0201, *2402
HLA-B*5101, *5102 or *5201
HLA-C none

HLA-DRB1*1501 or *1502

TABLE 5

| | DC | DC |
|---|---|---|
| | — | EBV-antigen |
| First stimulation | 0.11% | 3.56% |
| Second stimulation | 0.01% | 9.12% |
| Third stimulation | 0.01% | 24.52% |

### COMPARATIVE EXAMPLE 2

<Induction of Melanoma Antigen-Specific CTL Using Auto-DC>

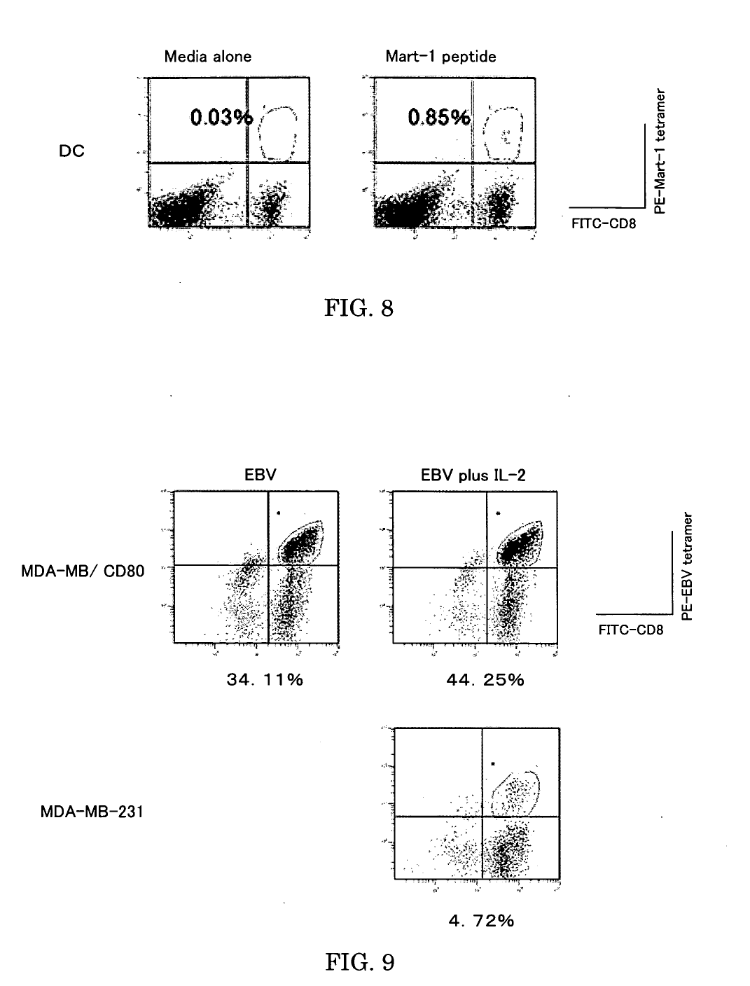

CD8-positive CTLs specific for a melanoma antigen were induced similarly to Comparative example 1 except for replacing the EBV antigenic peptide with the Mart-1 and replacing the EBV antigenic peptide-specific tetramer with the Mart-1-specific tetramer, and the induction efficiency thereof was measured by a flow cytometry. FIG. 8 shows an example of the measurement results, and Table 6 below describes the ratio of Mart-1-specific CTLs.

TABLE 6

| | DC | DC |
|---|---|---|
| | — | Mart-1 antigen |
| First stimulation | 0.03% | 0.85% |

<Results of Examples 2, 3 and Comparative Examples 1, 2>

As shown in FIGS. 5 to 8 and Tables 3 to 6 above, although the MDA-MB/CD80 did not express MHC class II molecules, which are essential molecules for known antigen-presenting abilities, this cell line was able to induce antigen-specific CTLs much more efficiently than the case of co-culturing with auto-DCs. Further, the co-culturing with auto-DCs now is considered to be the most efficient in inducing antigen-specific CTLs, but even after three stimulations, it was shown only to achieve an induction efficiency much lower than the case of providing two stimulations using the CD80-expressing cell line of the present invention.

In other words, by culturing lymphocytes while bringing the CD80-expressing cell line of the present application into contact with a specific disease antigenic peptide, it becomes possible to reduce the processes compared with the conventional case and further to considerably improve the efficiency of inducing specific CTLs.

Although EBV and melanoma were used in the above-described tests, there is no limitation to these particular antigens. The present invention can be applied to any disease antigenic peptides that can bind to MHC class I molecules and be presented thereon.

That is to say, as long as peptides are presented on MHC class I molecules, then T cells having T cell receptors recognizing these peptides can be recognized. This alone is not sufficient to promote activation and proliferation of the T cells. However, when CD80 expressed by the cell line of the present invention is incorporated as a co-stimulatory molecule, it binds to CD28 on the T cells and adds a co-stimulatory signal via the CD28, thereby effectively promoting the activation and proliferation of the T cells.

### EXAMPLE 4

<Additional Effect of IL-2 in CTL Induction Using CD80-Expressing Cell Line>

In the 6 to 8-day-culturing after the first stimulation described in Example 2, the ratio of CD8-positive antigen-specific CTLs induced in the T cells was measured similarly to Example 2 with respect to the case of adding IL-2 (500 U/ml) on the third day and the case of adding no IL-2. FIG. 9 shows an example of the measurement results, and Table 7 below describes the ratio of EBV-specific CTLs.

TABLE 7

| | MDA-MB/CD80 | MDA-MB/CD80 | MDA-MB |
|---|---|---|---|
| | EBV-antigen | EBV-antigen | EBV-antigen |
| | IL-2 | — | IL-2 |
| First stimulation | 44.25% | 34.11% | 4.72% |

As shown in FIG. 9 and Table 7 above, even when the same amount of IL-2 was added, the MDA-MB/CD80_{G418} cell line showed about ten times as high an induction efficiency as the the MDA-MB-231 cell line expressing no CD80. Additionally, even the same CD80-expressing cell lines showed different induction efficiencies, that is, the one to which IL-2 was added achieved an improved specific CTL induction efficiency compared with the other without IL-2.

### EXAMPLE 5

<Establishment of cell Line Stably Expressing CD80: MDA-MB/CD80 Zeocin>

(1) Cloning of CD80 Gene Fragment

Similarly to Example 1, a CD80 gene fragment was amplified by a PCR method and introduced into plasmid pZeoSV2, thus obtaining pZeoSV2/CD80. When the sequence of the inserted human CD80 cDNA was analyzed by a DNA sequencer, it perfectly matched with the sequence of human CD80 cDNA reported in the paper [Freeman G. J. et al. (J. Immunol.) 143 2714-2722 (1989)].

(2) Establishment of Cell Line Stably Expressing CD80

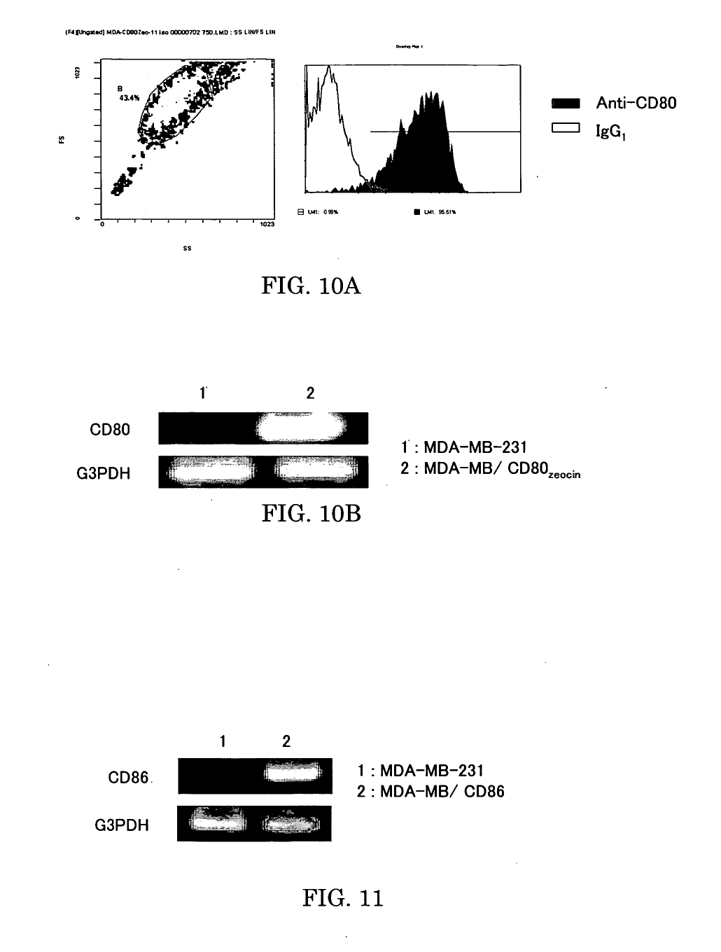

A cell line MDA-MB/CD80 zeocin expressing human CD80 stably and constantly in a MDA-MB-231 cell was established similarly to Example 1 except for using the above-noted pZeoSV2/CD80 as plasmid and zeocin as the antibiotic for selection. The expression of CD80 of the resultant cell line MDA-MB/CD80 zeocin was confirmed by detecting CD80 on the cell surface by a flow cytometry and detecting the expression of CD80 mRNA by a RT-PCR method. FIGS. 10A and 10B show examples of these results. FIG. 10A shows an analysis result in the flow cytometry, while FIG. 10B shows the result of RT-PCR. In FIG. 10B, lane 1 indicates MDA-MB-231, which is a parent cell line of the MDA-MB/CD80 zeocin, and lane 2 indicates the MDA-MB/CD80 zeocin. G3PDH was expressed in every cell and served as a marker used for showing that the total RNA amounts used for RT-PCR were equal. As shown in FIGS. 10A and 10B, it was confirmed that the cell line MDA-MB/CD80 zeocin stably expressing CD80 was established.

### EXAMPLE 6

<Establishment of Cell Line Stably Expressing CD86: MDA-MB/CD86>

(1) Cloning of CD86 Gene Fragment

A CD86 gene fragment was amplified by a PCR method. A cDNA library of peripheral blood lymphocyte of a normal subject was used as a template, and the two DNA oligomers listed below synthesized based on the CD86 cDNA sequence in the document [Proc. Natl. Acad. Sci. U.S.A. 99 (26), 16899-16903 (2002)] were used as primers. The underlined sequences in the primer sequences below respectively indicate the sequences Nhe I (CD86: F) and Xba I (CD86: R) of a restriction site to be inserted into a vector.

| | |
|---|---|
| CD86: F | |
| 5′-TAAGCTAGCACCATGGATCCCCAGTGCACTATG | (SEQ ID NO: 5) |
| GGACTGAG-3′ | |
| CD86: R | |
| 5′-TAACTCGAGTTAAAAACATGTATCACTTTTGTC | (SE ID NO: 6) |
| GCATG-3′ | |

The PCR method was carried out under the same reaction condition as Example 1. The DNA fragment specifically amplified by the PCR method was isolated by 1.5% agarose gel electrophoresis, treated with restriction enzymes Nhe I and Xba I, and inserted between Nhe I and Xba I sites of plasmid pZeoSV2 (manufactured by Invitrogen Corporation), thus obtaining pZeoSV2/CD86. When the sequence of the inserted CD86 cDNA was analyzed by a DNA sequencer, it perfectly matched with the sequence of CD86 cDNA reported in the above-mentioned document.

(2) Establishment of Cell Line Stably Expressing CD86

Using a laboratory dish with a diameter of 35 mm, MDA-MB-231 cells were cultured in RPMI 1640 medium containing 10% fetal bovine serum for 4 to 6 hours. Into these cells, 4 μg of the pZeoSV/CD86 was transfected by a lipofection method. 48 hours after the transfection, a transient expression of CD86 was confirmed by a RT-PCR method. FIG. 11 shows the result. As shown in this figure, it was confirmed that mRNA of CD86 was expressed at a high level. These transient CD86-expressing cell lines make it possible to obtain a cell line MDA-MB/CD86 expressing human CD86 stably at a high level by replacing the medium with a medium containing an antibiotic zeocin, selecting cells with chromosome into which the plasmid was integrated and further cloning from a single cell of the selected cells by a limiting dilution method.

### EXAMPLE 7

<Induction of EBV Antigen-Specific CTL Using Transiently Transfected CD86 Cell Line>

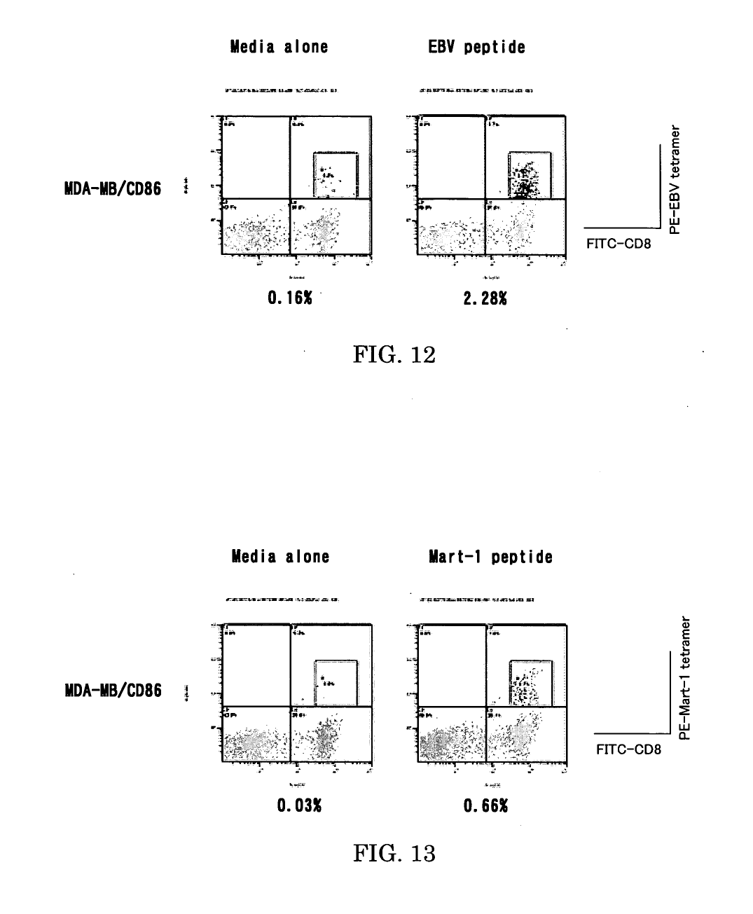

CD8-positive CTLs specific for the EBV antigen were induced similarly to Example 2 except for replacing the MDA-MB/CD80 with the transiently transfected CD86 cell line produced in Example 6, and the induction efficiency thereof was measured by a flow cytometry. FIG. 12 shows an example of the measurement results after the first stimulation, and Table 8 below describes the ratio of EBV-specific CTLs.

TABLE 8

| | MDA-MB/CD86 | MDA-MB/CD86 |
|---|---|---|
| | — | EBV antigen |
| First stimulation | 0.16% | 2.28% |

### EXAMPLE 8

<Induction of Melanoma Antigen-Specific CTL Using Transiently Transfected CD86 Cell Line>

CD8-positive CTLs specific for the melanoma antigen were induced similarly to Example 7 except for replacing the EBV antigenic peptide with a melanoma peptide (Mart-1), which was a cancer antigenic peptide, and replacing the EBV antigenic peptide-specific tetramer with a Mart-1-specific tetramer (T-Select MHC Tetramer, manufactured by Medical & Biological Laboratories Co., Ltd.). The induction efficiency thereof was measured by a flow cytometry. FIG. 13 shows an example of the measurement results after the first stimulation, and Table 9 below describes the ratio of Mart-1-specific CTLs.

TABLE 9

| | MDA-MB/CD80 | MDA-MB/CD80 |
|---|---|---|
| | — | Mart-1 antigen |
| First stimulation | 0.03% | 0.66% |

<Results of Examples 7 and 8>

As shown in FIGS. 12, 13 and Tables 8, 9, even when using CD86 instead of CD80 as a co-stimulatory molecule, it was shown that CD8-positive antigen-specific CTLs could be induced efficiently.

### EXAMPLE 9

<Establishment of Cell Line Stably Expressing CD80: TUHR10TKB/CD80>

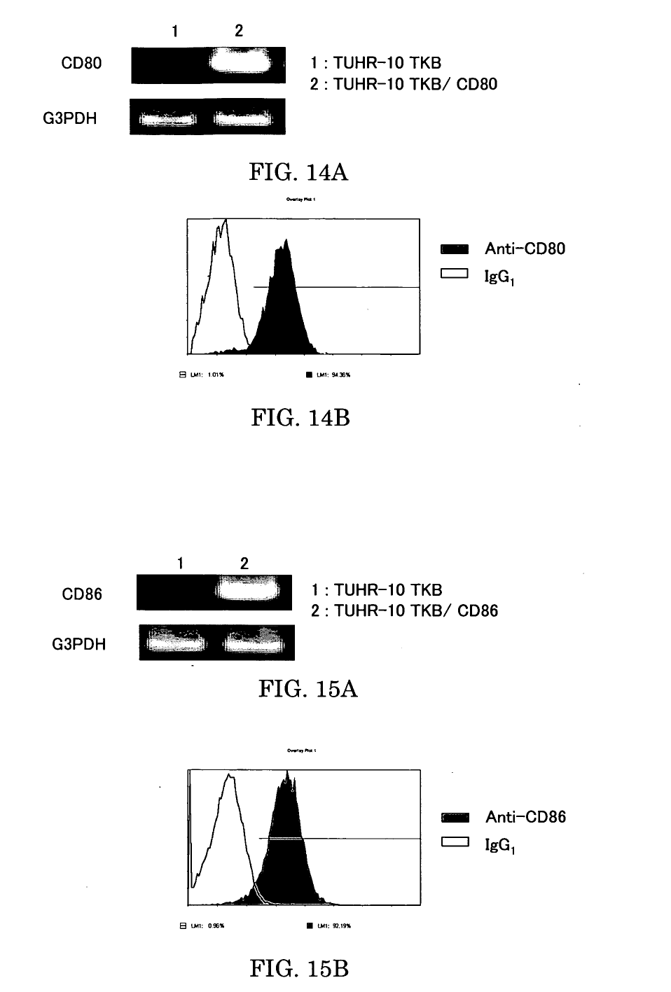

Using a laboratory dish with a diameter of 35 mm, cells of a cell line TUHR10TKB whose MHC class I type was HLA-A2/24 were cultured in RPMI 1640 medium containing 10% fetal bovine serum for 4 to 6 hours. Into these cells, 4 μg of the pZeoSV2/CD80 was transfected by a lipofection method. 48 hours after the transfection, a transient expression of CD80 was confirmed by a RT-PCR method. FIG. 14A shows the result. As shown in this figure, it was confirmed that mRNA of CD80 was expressed at a high level.

Then, a cell line TUHR10TKB/CD80 expressing human CD80 stably at a high level was obtained by replacing the medium with a medium containing an antibiotic zeocin, selecting cells with chromosome into which the plasmid was integrated, and further cloning from a single cell of the selected cells by a limiting dilution method. The expression of CD80 of the resultant cell line TUHR10TKB/CD80 was confirmed by detecting CD80 on the cell surface by a flow cytometry. FIG. 14B shows an analysis result in the flow cytometry. As shown in FIG. 14B, it was confirmed that the cell line TUHR10TKB/CD80 stably expressing CD80 was established.

The HLA types of the TUHR10TKB cells used here were as follows.
HLA-A*0201, HLA-A*2402
HLA-B*4001, HLA-B*4002
HLA-C*0303, HLA-C*0304

### EXAMPLE 10

<Establishment of Cell Line Stably Expressing CD86: TUHR10TKB/CD86>

Using a laboratory dish with a diameter of 35 mm, cells of the TUHR10TKB were cultured in RPMI 1640 medium containing 10% fetal bovine serum for 4 to 6 hours. Into these cells, 4 μg of the pZeoSV2-CD86 was transfected by a lipofection method. 48 hours after the introduction, a transient expression of CD86 was confirmed by a RT-PCR method. FIG. 15A shows the results. As shown in this figure, it was confirmed that mRNA of CD86 was expressed at a high level.

Then, a cell line TUHR10TKB/CD86 expressing human CD86 stably at a high level was obtained by replacing the medium with a medium containing an antibiotic zeocin, selecting cells with chromosome into which the plasmid was integrated, and further cloning from a single cell of the selected cells by a limiting dilution method. The expression of CD86 of the resultant cell line TUHR10TKB/CD86 was confirmed by detecting CD86 on the cell surface by a flow cytometry. FIG. 15B shows an analysis result in the flow cytometry. As shown in FIG. 15B, it was confirmed that the cell line TUHR10TKB/CD86 stably expressing CD86 was established.

As described above, the method for inducing CTLs according to the present invention can induce CTLs specific for an antigen associated with a certain disease in a more quick, simplified and precise manner than a conventional inducing method using antigen-presenting cells such as DCs. The amount of induced CTLs is greater in the present invention than the conventional method. Consequently, the present invention is useful in the field of pharmaceutical preparations such as drugs or vaccines for cancers and viral infections and the field of medicine such as cell-mediated immunotherapies.

The invention may be embodied in other specific forms without departing from the spirit or essential characteristics thereof. The embodiments disclosed in this application are to be considered in all respects as illustrative and not restrictive, the scope of the invention being indicated by the appended claims rather than by the foregoing description, all changes that come within the meaning and range of equivalency of the claims are intended to be embraced therein.

Incorporation by Reference.

Each document, patent application or patent publication cited by or referred to in this disclosure is incorporated by reference in its entirety. Any patent document to which this application claims priority is also incorporated by reference in its entirety. Specifically, priority documents JP patent application No. 2003-341822, filed on Aug. 25, 2003, and JP patent application No. 2004-020436, filed on Jan. 28, 2004 are hereby incorporated by reference.

1. A method for inducing a CTL (cytotoxic T lymphocyte), comprising:
contacting a cell line that expresses at least one major histocompatibility antigen (MHC) class I molecule and which has been transformed with at least one co-stimulatory molecule with an isolated or purified antigenic peptide and with a lymphocyte for a time and under conditions suitable for inducing a cytotoxic lymphocyte (CTL) specific for said antigenic peptide.

2. The method of claim 1, wherein said contacting occurs in vitro.

3. The method of claim 1, wherein said cell line is a human cell line, which expresses at least one MHC class I antigen heavy chain and β2-microglobulin, and at least one co-stimulatory, accessory, or adhesion molecules.

4. The method of claim 1, wherein said cell line is a stomach cancer-derived JR-st, a renal cancer-derived TUHR10TKB or a breast cancer-derived MDA-MB-231.

5. The method of claim 1, wherein said cell line has been transformed with a nucleic acid expressing CD80 (B7.1).

6. The method of claim 5, wherein said cell line expresses CD80, CD54 (ICAM-1) and CD40.

7. The method of claim 1, wherein said cell line has been transformed with a nucleic acid encoding CD86 (B7.2).

8. The method of claim 7, wherein said cell line expresses CD86 (B7.2), CD54 and CD40.

9. The method of claim 1, wherein said antigenic peptide consists of a peptide having 8-11 amino acid residues.

10. The method of claim 1, wherein said-antigenic peptide corresponds to a portion of a cancer antigen.

11. The method of claim 1, wherein said antigenic peptide corresponds to a portion of an antigen associated with an infectious disease.

12. The method of claim 1, wherein said cell line expresses a class I major histocompatibility antigen which is HLA-A2 or HLA-A24, or both.

13. The method of claim 1, wherein said cell line is homozygous for HLA-A2.

14. The method of claim 1, wherein said cell line is homozygous for HLA-A*0201.

15. The method of claim 1, wherein said cell line is homozygous for HLA-A24.

16. The method of claim 1, wherein said cell line is homozygous for HLA-A*2401.

17. The method of claim 1, wherein said cell line is homozygous for HLA-A*2402.

18. The method of claim 1, wherein said lymphocyte is an autologous lymphocyte or an allogeneic lymphocyte that shares at least one MHC class I molecule on said cell line.

19. An antigen-specific cytotoxic lymphocyte produced by the method of claim 1.

20. An antigen-specific cytotoxic lymphocyte produced by the method of claim 4.

21. An antigen-specific cytotoxic lymphocyte produced by the method of claim 5.

22. An antigen-specific cytotoxic lymphocyte produced by the method of claim 7.

23. A method for treating or preventing cancer or an infectious disease comprising administering to a subject in need thereof the CTL produced by the method of claim 1.

24. A method for treating or preventing cancer or an infectious disease comprising administering to a subject in need thereof the CTL produced by the method of claim 4.

25. The method of claim 23, wherein said CTL is produced in vitro and is infused into said patient.

26. The method of claim 23, wherein said disease is cancer and said CTL recognizes an antigenic determinant of an antigen associated with the cancer.

27. The method of claim 23, wherein said disease is an infectious disease, and said CTL recognizes an antigenic determinant of an antigen associated with said infectious disease.

28. A method for inducing a CTL, comprising:
contacting with a lymphocyte with a cell line that expresses at least one major histocompatibility antigen (MHC) class I molecule, an antigen and a co-stimulatory molecule for a time and under conditions suitable for induction of a cytotoxic lymphocyte (CTL) specific for said antigen, wherein said cell line has been transformed with at least one co-stimulatory molecule or exogenous antigen.

29. The method of claim 28, wherein said contacting occurs in vitro.

30. The method of claim 28, wherein said cell line is a human cell line, which expresses at least one MHC class I antigen heavy chain and β2-microglobulin, and at least one co-stimulatory or accessory molecules such as adhesion molecules.

31. The method of claim 28, wherein said cell line is a stomach cancer-derived JR-st, a renal cancer-derived TUHR10TKB or a breast cancer-derived MDA-MB231.

32. The method of claim 28, wherein said cell line has been transformed with a nucleic acid expressing CD80 (B7.1).

33. The method of claim 32, wherein said cell line expresses CD80, CD54 (ICAM-1) and CD40.

34. The method of claim 28, wherein said cell line has been transformed with a nucleic acid encoding CD86 (B7.2).

35. The method of claim 34, wherein said cell line expresses CD86 (B7.2), CD54 and CD40.

36. The method of claim 28, wherein said antigen is a cancer antigen.

37. The method of claim 28, wherein said antigen is an antigen associated with an infectious disease.

38. The method of claim 28, wherein said cell line expresses a class I major histocompatibility antigen which is HLA-A2 or HLA-A24, or both.

39. The method of claim 28, wherein said cell line is homozygous for HLA-A2.

40. The method of claim 28, wherein said cell line is homozygous for HLA-A*0201.

41. The method of claim 28, wherein said cell line is homozygous for HLA-A24.

42. The method of claim 28, wherein said cell line is homozygous for HLA-A*2401.

43. The method of claim 28, wherein said cell line is homozygous for HLA-A*2402.

44. The method of claim 28, wherein said lymphocyte is an autologous lymphocyte or an allogeneic lymphocyte that shares at least one MHC class I molecule with said cell line.

45. An antigen-specific cytotoxic lymphocyte produced by the method of claim 28.

46. An antigen-specific cytotoxic lymphocyte produced by the method of claim 31.

47. An antigen-specific cytotoxic lymphocyte produced by the method of claim 32.

48. An antigen-specific cytotoxic lymphocyte produced by the method of claim 34.

49. A method for treating or preventing cancer or an infectious disease comprising administering to a subject in need thereof the CTL produced by the method of claim 28.

50. A method for treating or preventing cancer or an infectious disease comprising administering to a subject in need thereof the CTL produced by the method of claim 31.

51. The method of claim 49, wherein said CTL is produced in vitro and is infused into said patient.

52. The method of claim 49, wherein said disease is cancer and said CTL recognizes an antigenic determinant of an antigen associated with the cancer.

53. The method of claim 49, wherein said disease is an infectious disease, and said CTL recognizes an antigenic determinant of an antigen associated with said infectious disease.

54. A method for testing a proliferation potential of a CTL, comprising:
co-culturing a cell line that expresses at least one MHC class I molecule and a co-stimulatory molecule with a lymphocyte harvested from a subject, and
simultaneously or subsequently bringing the cell line into contact with a disease antigenic peptide, thus determining the ability of said cell line and antigenic peptide to induce proliferation of a CTL.

55. The method for testing a proliferation potential of a CTL according to claim 54, wherein said cell line is a stomach cancer-derived JR-st, a renal cancer-derived TUHR10TKB or a breast cancer-derived MDA-MB-231.

56. The method for testing a proliferation potential of a CTL according to claim 54, wherein the co-stimulatory molecule on said cell line is at least one of CD80 and CD86.

57. The method for testing a proliferation potential of a CTL according to claim 54, wherein the type of the MHC class I of the cell line is HLA-A2 or HLA-A24, or both.

58. The method for testing a proliferation potential of a CTL according to claim 54, wherein the antigenic peptide is a cancer antigenic peptide or an infectious disease antigenic peptide.

59. A method for testing a proliferation potential of a CTL, comprising:
co-culturing a cell line that expresses at least one MHC class I molecule with a lymphocyte harvested from a subject, a disease antigen and a co-stimulatory molecule.

60. The method for testing a proliferation potential of a CTL according to claim 59, wherein said cell line is a stomach cancer-derived JR-st, a renal cancer-derived TUHR10TKB or a breast cancer-derived MDA-MB-231.

61. The method for testing a proliferation potential of a CTL according to claim 59, wherein the co-stimulatory molecule on the cell line is at least one of CD80 and CD86.

62. The method for testing a proliferation potential of a CTL according to claim 59, wherein the type of the MHC class I of the cell line is HLA-A2 or HLA-A24, or both.

63. The method for testing a proliferation potential of a CTL according to claim 59, wherein the antigen is a cancer antigen or an infectious disease antigen.
